(19) United States
(12) Patent Application Publication
(10) Pub. No.: US 2009/0137565 A1
(43) Pub. Date: May 28, 2009

(54) Method for treatment of movement disorders

(75) Inventor: Steven Frucht, New York, NY (US)

Correspondence Address: WilmerHale/Columbia University, 399 PARK AVENUE, NEW YORK, NY, 10022, US

(73) Assignee: The Trustees of Columbia University in the City of New York, New York, NY (US)

(21) Appl. No.: 11/667,530

(22) PCT Filed: Nov. 9, 2005
(86) PCT No.: PCT/US05/40877

§ 371 Date: Jul. 14, 2008

Related U.S. Application Data
(60) Provisional application No. 60/626,645, filed on Nov. 10, 2004.

(51) Int. Cl. A61K 31/19; A61K 31/5513; A61K 31/515; A61P 25/08

(52) U.S. Cl. 514/221; 514/557; 514/270

(57) ABSTRACT

The invention is directed to methods of treating movement disorders by administering an effective amount of the compound of formula (I) to patients in need thereof. More particularly, the invention is directed to a method for treating myoclonus including administering to a patient a compound of formula (I), wherein the myoclonus is not alcohol responsive essential myoclonus with dystonia. In some embodiments, the myoclonus is posthypoxic myoclonus. The invention is also directed to a method for treating dystonia, essential tremor cerebellar tremor, a tic, or chorea, including administering to a patient a compound of formula (I).

This application claims priority of U.S. Provisional patent application Ser. No. 60/626,645, filed Nov. 10, 2004, which is hereby incorporated by reference in its entirety.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

This invention relates to methods for treatment of movement disorders such as hyperkinetic movement disorders, tics, and chorea. More particularly, the invention relates to treatment of myoclonus, dystonia and essential tremor by administrating compounds of formula (I), defined herein. Such compounds include sodium gamma-hydroxybutyrate (Xyrem®).

### BACKGROUND OF THE INVENTION

Movement disorders encompass a wide variety of neurological conditions affecting motor control and muscle tone. These conditions are typified by the inability to control certain bodily actions. Accordingly, these conditions pose a significant quality of life issue for patients. Nonlimiting examples of movement disorders include Parkinson's syndrome, dyskinesias, dystonias, myoclonus, chorea, tics, and tremor.

Dystonia, one type of movement disorder, is a neurological disorder characterized by sustained, involuntary movements. These movements typically produce twisting postures. Dystonia is also known as torsion dystonia. A large number of conditions produce dystonia, including genetic causes, toxin or drug-induced causes, and degenerative illnesses in which dystonia is manifested.

Essential tremor is another type of movement disorder, separate from dystonia. It is the most common cause of tremor in the adult population, affecting approximately five to ten million Americans. Patients with essential tremor exhibit involuntary, rhythmic tremor, or shaking, of a body part. Commonly, essential tremor affects the hands, head, or voice; but it can also affect the tongue, legs, or trunk. The tremor of one body part can occur alone or in combination with other body parts. Depending on its severity, essential tremor can escalate from being merely a slight disturbance to a functional disability and physical handicap. Especially where tasks involve fine motor control, patients with essential tremor may have difficulty performing these skills. For example, a severe tremor in the hands makes eating, drinking, writing, and dressing difficult. Tremors associated with essential tremor typically worsen over time. While the exact cause of essential tremor is not known, it is often inherited.

Myoclonus is yet another form of movement disorder, characterized by very fast, lightning-like jerks, caused by brief, sudden muscle contractions (positive myoclonus) or relaxations (negative myoclonus). The shock-like involuntary movements of myoclonus are often severe enough to interfere with the basic activities of daily living. These jerks may affect any part of the body. Some myoclonic movements occur in response to stimulus, while others occur when making a movement. Still other myoclonic movements occur spontaneously. Myoclonus occurs as a result of any number of conditions affecting the central nervous system, including genetic disorders (e.g., essential myoclonus), drug and toxin-induced conditions, after cardiac arrest (e.g., posthypoxic myoclonus), and as the result of progressive neurological degenerative disorders (e.g., multiple sclerosis, Alzheimer's disease or Creutzfeldt-Jakob disease). For example, myoclonus with dystonia, also known as essential myoclonus, is a rare, genetic form of myoclonus. A subset of myoclonus with dystonia, alcohol responsive myoclonus with dystonia is completely resistant to all other treatments and is exquisitely responsive to alcohol.

Another type of myoclonus, posthypoxic myoclonus, is an often-devastating, rare neurologic disorder that follows an episode of oxygen deprivation to the brain, such as following cardiac or respiratory arrest, or following kidney or liver failure. Some patients who survive such trauma have normal mentation but develop severe involuntary movements. Attempts to perform manual tasks or to walk typically trigger intractable action and intention myoclonus. Negative myoclonic jerks often affect muscles of postural support, producing a characteristic bouncing gait which may render a patient wheelchair-bound. Both cortical and subcortical foci may be responsible for generating myoclonic jerks. In a recent study using positron emission tomography, a characteristic pattern of ventrolateral thalamic activation in posthypoxic patients that was absent in controls was demonstrated. Treatment of posthypoxic myoclonus relies on medications, which are only partially effective. Treatment with anti-myoclonic agents such as clonazepam, valproic acid, levetiracetam or zonisamide is sometimes helpful, however many patients benefit incompletely and others are left in a totally dependent state.

Movement disorders, such as myoclonus, dystonia, chorea, tics, and essential tremor are treated with benzodiazepines, anticonvulsants and β-adrenergic blockers. Other therapies used for movement disorders, particularly dystonia, include anticholinergics and dopamine-blocking or deleting agents. The effectiveness of these agents is diminished, however, because not all patients respond well to the drug therapy, some of the drugs are not well tolerated, and the drugs may cause undesirable side effects. Additionally, in some cases, patients develop tolerance to the drugs, requiring ever increasing dosages to ameliorate the symptoms. Surgery, such as thalamotomy pallidotomy and deep brain stimulation, are also used to treat hyperkinetic movement disorders. However, because of their invasive nature, these treatment options are less desirable. Therefore, a need exists for alternative drag-based therapies for movement disorders such as hyperkinetic movement disorders that do not pose the drawbacks of the present therapies.

Several reports have described patients with progressive myoclonic epilepsy and posthypoxic myoclonus experiencing improvement of myoclonus with administration of alcohol. Rare patients with posthypoxic myoclonus have been described in which the myoclonus dramatically improves with ingestion of alcohol. This effect may be striking and is typically short-lived, lasting only hours. Similarly, essential tremor is also frequently responsive to alcohol. Although an appealing therapeutic option, ingestion of alcohol is ill advised in patients who may already be sedated from concomitant anti-epileptic medications. Alcohol's ability to lower the seizure threshold is also of concern in patients who have a history of seizures. Moreover, alcohol produces a euphoric effect at the doses typically needed to produce relief of tremor or myoclonus. Further drawbacks to treatment with alcohol include gastroesophageal erosion with chronic use, increased risk of liver toxicity including the possibility of cirrhosis, caloric and sugar intake which may be contraindicated in patients with obesity and diabetes, and health concerns in patients with cardiac disease. Finally, there is a probability that continued use of alcohol to treat movement disorders may lead to a rebound effect, where the movement disorder symptoms return with increased severity when the alcohol wears off. A single patient with alcohol-responsive myoclonus with dystonia was recently reported to display improved myoclonus when treated with sodium gamma-hydroxybutyrate, an approved medication that is similar to alcohol in its effects on the nervous system.

Therefore, a need exists for effective drug-based treatments of movement disorders, particularly myoclonus, dystonia, chorea, tics, and essential tremor.

### SUMMARY OF THE INVENTION

The present invention provides methods of treating involuntary movement disorders that are presently untreatable, or inadequately treated by best medical therapy. The present invention provides a therapeutic method to treat movement disorders comprising administering a compound of formula (I):

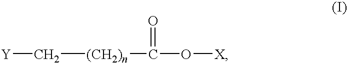

wherein n is 1-2, X is H, a pharmaceutically acceptable cation or (C₁-C₄)alkyl, and Y is hydroxy, (C₁-C₄)alkoxy, CH(Z)CH₃, (C₁-C₄)alkanoyloxy, phenylacetoxy, or benzoyloxy or where X and Y are connected by a single bond, wherein Z is hydroxy, (C₁-C₄)alkoxy, (C₁-C₄)alkanoyloxy, phenylacetoxy, or benzoyloxy.

In one embodiment, the movement disorder is a hyperkinetic movement disorder such as myoclonus. In another embodiment, the myoclonus is not alcohol responsive myoclonus with dystonia. In further embodiments, the myoclonus is posthypoxia myoclonus or is not alcohol responsive posthypoxia myoclonus. In yet another embodiment, the movement disorder is essential tremor. The amount of one or more of the compounds of formula (I) is effective to eliminate or alleviate at least one of the symptoms of myoclonus or essential tremor. Such symptoms include, but are not limited to negative myoclonus, myoclonus at rest, stimulus-sensitive myoclonus, action myoclonus, benign tremor, postural tremor, and kinetic tremor.

The present invention also provides a therapeutic method to treat hyperkinetic movement disorders including administering to a human afflicted with a myoclonus an effective amount of a compound of formula (I), wherein the amount is effective to alleviate at least one symptom of the myoclonus, wherein the myoclonus is not alcohol-sensitive essential myoclonus with dystonia. Nonlimiting examples of myoclonus include palatal myoclonus, a startle syndrome, and spinal myoclonus.

The present invention further provides a therapeutic method to treat a hyperkinetic movement disorder including administering to a human afflicted with a dystonia, essential tremor, cerebellar tremor, a tic, chorea (such as Huntington's disease), ballismus, progressive myoclonic epilepsy, focal task-specific dystonia, and brainstem myoclonus an effective amount of a compound of formula (I), wherein the amount is effective to alleviate at least one symptom of the myoclonus, wherein the myoclonus is not alcohol-sensitive essential myoclonus with dystonia. Nonlimiting examples of dystonia include generalized dystonia and focal dystonia.

Preferred compounds of formula (I) include the alkali metal salts of 4-hydroxybutyric acid (n=2), such as 4-hydroxybutanoic acid monosodium salt (GHB), γ-butyrolactone and the (C₁-C₂) alkyl esters of 4-acetoxybutanoic acid or 4-benzoyloxybutanoic acid. Other compounds that can be used in the present methods include those disclosed in Kluger (U.S. Pat. Nos. 4,599,355 and 4,738,985). Other prodrugs of gamma-hydroxyburic acid are also useful in the practice of the invention, including 1,4-butane diol, 4-hydroxyvaleric acid, γ-valeroacetone, trans-4-hydroxy-crotonic acid, 4-methyl-4hydroxycrotonic acid, and trans-4-phenyl-hydroxycrotonic acid and their salts. For further analogs of gamma-hydroxyburic acid contemplated by the invention, see J. J. Bourguignon, et al., J. Med. Chem. 31(5):893-897 (May 1988).

In one embodiment of the invention, treatment of patients with sodium oxybate (Xyrem®) alleviates symptoms of movement disorders, particularly hyperkinetic movement disorders such as myoclonus, dystonia, chorea, tics, and essential tremor. In particular, this effect was observed in a patient with severe posthypoxic myoclonus whose movements were refractory to all available anti-myoclonic agents.

### BRIEF DESCRIPTION OF THE FIGURES

FIG. 1 is a graphical representation of Unified Myoclonus Rating Scale scores as a function of daily dose of Xyrem®.

FIG. 2 is a photograph of a patient after treatment using chopsticks at the beginning of her treatment with 4 gm of Xyrem® twice daily.

FIG. 3 is a photograph of a letter written by the patient shown in FIG. 2, on her own initiative, and without assistance, after treatment with Xyrem®.

FIG. 4 depicts spirals drawn by patients having ET before and after receiving sodium oxybate. Pre- and post-treatment spirals are shown for patient #4 (PME), (A: pre-treatment; B: post-treatment (2.5 gm TID)), and patient #18 (ET), (C: pre-treatment; D: post-treatment 3 gm TID).

### DETAILED DESCRIPTION OF THE INVENTION

The patent and scientific literature referred to herein establishes knowledge that is available to those with skill in the art. The issued patents, applications, and other publications that are cited herein are hereby incorporated by reference to the same extent as if each was specifically and individually indicated to be incorporated by reference. In the case of inconsistencies, the present disclosure will prevail.

The following publications are hereby incorporated by reference in their entirety: U.S. Pat. Nos. 5,990,162, 6,472,431, and 6,780,889 and U.S. Patent Publication No. 2004/0092455.

### DEFINITIONS

For purposes of the present invention, the following definitions will be used:

The term “carrier” is used herein to refer to a pharmaceutically acceptable vehicle for a pharmacologically active agent. The carrier facilitates delivery of the active agent to the target site without terminating the function of the agent. Nonlimiting examples of suitable forms of the carrier include solutions, creams, gels, gel emulsions, jellies, pastes, lotions, salves, sprays, ointments, powders, solid admixtures, aerosols, emulsions (e.g., water in oil or oil in water), gel aqueous solutions, aqueous solutions, suspensions, liniments, tinctures, and patches suitable for topical administration.

The term “pharmaceutically acceptable” is used herein to mean suitable for use in mammals. Pharmaceutically acceptable salts of a compound include acid and base addition salts thereof. Suitable acid addition salts are formed from acids that form non-toxic salts. Nonlimiting examples include hydrochloride, hydrobromide, hydroiodide, nitrate, sulfate, bisulfate, phosphate, acid phosphate, acetate, lactate, citrate, acid citrate, tartrate, bitartrate, succinate, maleate, fumarate, gluconate, saccharate, benzoate, methanesulfonate, ethanesulfonate, benzenesulfonate, p-toluenesulfonate and pamoate (i.e., 1,1-methylene-bis-(2-hydroxy-3-naphthoate)) salts. Suitable base salts are formed from bases which form non-toxic salts. Nonlimiting examples include aluminum, sodium, potassium, calcium, magnesium, zinc and diethanolammonium salts. For a review of suitable salts, see, e.g., Berge et al., J. Pharm. Sci. 66:1-19 (1977) and Remington: The Science and Practice of Pharmacy, 20th Ed., ed. A. Gennaro, Lippincott Williams & Wilkins, 2000.

Pharmaceutically acceptable esters include those esters that retain, upon hydrolysis of the ester bond, the biological effectiveness and properties of the carboxylic acid and are not biologically or otherwise undesirable. For a description of pharmaceutically acceptable esters as prodrugs, see Bundgaard, E., ed., Design of Prodrugs, Elsevier Science Publishers, Amsterdam, 1985. These esters are typically formed from the corresponding carboxylic acid and an alcohol. Generally ester formation can be accomplished via conventional synthetic techniques. (See, e.g., March's Advanced Organic Chemistry, 3^{rd} Ed., John Wiley & Sons, New York p. 1157, 1985 and references cited therein, and Mark et al., Encyclopedia of Chemical Technology, John Wiley & Sons, New York, 1980.) The alcohol component of the ester will generally comprise (i) a C₂-C₁₂ aliphatic alcohol that optionally contains one or more double bonds and optionally contains branched carbons or (ii) a C₇-C₁₂ aromatic or heteroaromatic alcohol. This invention also contemplates the use of those compositions which are both esters as described herein and at the same time are the pharmaceutically acceptable salts thereof.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of ≦20%.

The term “effective” is used herein to indicate that the active agent is administered in an amount and at an interval that results in the desired treatment or improvement in the disorder or condition being treated (e.g., an amount effective to decrease at least one myoclonic or tremor symptom).

Gamma-hydroxybutyric acid (GHB) is a naturally occurring substance found in the human nervous system and other organs. It is found in highest concentrations in the hypothalamus and basal ganglia. The discovery of central recognition sites with high affinity for this metabolite suggests that it functions as a neurotransmitter or neuromodulator rather than as an incidental breakdown product of gamma-aminobutyric acid metabolism.

GHB is a short-chain fatty acid, structurally closely related to gamma-aminobutyric acid (GABA). An endogenous inhibitory neurotransmitter in the mammalian brain, GHB occurs naturally at concentrations of 1-4 micromolar. Unlike other neurotransmitters it is able to pass through the blood-brain barrier, and exogenously administered drug significantly raises the concentration within the brain. Wong C. G. T., et al., Trends in Pharmacological Sciences 25:29-34 (2004); Waszkielewicz A., et al., Pol. J. Pharmacol. 56:43-49 (2004). GABA is the precursor of GHB, and it is synthesized in presynaptic neuronal terminals. A specific, high-affinity receptor for GHB is present in the brain in highest density in the hippocampus, cortex and thalamus. GHB may act at both its own receptor and at the GABA-B receptor, directly stimulating the former or indirectly the latter after undergoing conversion to GABA. Id.

The sodium salt of GHB was previously classified as a schedule I agent as the result of its unfortunate illicit manufacture and abuse. However it has been legitimately used in Europe for years for the maintenance of alcohol abstinence and withdrawal. Moncini M., et al., Alcohol 20:285-291 (2000); Korninger C., et al., Acta Med. Austriaca 30:83-86 (2003); Addolorato G., et al., Drug Alcohol Depend 53:7-10 (1998). More recently, sodium gamma-hydroxybutyric acid has been reclassified as a schedule III agent in the United States, under the name Xyrem® (Orphan Medical, Inc., Minnetonka, Minn.). Xyrem® is approved specifically for use in narcoleptic patients with cataplexy. All patients treated with Xyrem® in the United States are enrolled in the Xyrem® Success Program, a safety and monitoring system that ensures that the drug is being used and handled appropriately. Fuller D. E., et al., Drug Saf 27:293-306 (2004). When administered correctly, the drug is safe and well tolerated. Sleep 25:42-49 (2002).

Xyrem® (sodium oxybate) is a central nervous system depressant with anti-cataplectic activity. Xyrem® is a white powder that is given by mouth in liquid form, dissolved in water. It is metabolized to carbon dioxide and water, has no active metabolites and does not alter the activity of the cytrochrome P450 system. A total of 448 patients with narcolepsy received Xyrem® in clinical trials. For patients with cataplexy, the approved indication for Xyrem, the drug is given at a starting dose of 4.5 gm per night in two equally divided doses. The first dose of Xyrem® is given at bedtime, several hours after a meal, and the second dose is given 2½ to 4 hours later (the patient is awakened). The dose may be increased to a maximum of 9 gm per day over eight weeks in increments of 1.5 g per day.

In healthy human volunteers, about 30-60 mg/kg doses of 4-hydroxybutanoic acid monosodium salt (sodium oxybate or GHB; Merck Index 8603) promote a normal sequence of NREM and REM sleep lasting about 2-3 hours. The most consistent effect observed in patients after GHB administration is an increase in Slow Wave sleep (SWS). Total nocturnal REM sleep duration is usually unchanged. Total sleep time at night may be increased or unchanged. Narcoleptic patients have not shown tolerance to the hypnotic actions of GHB over a 6-month period.

Studies by R. Broughton and M. Mamelak, Can. J. Neur. Sci., 7:23 (1980), L. Scrima et al., Sleep, 13:479 (1990), and M. B. Scharf et al., Am. Fam. Phys., 143 (July 1988) have evaluated the effects of GHB in the treatment of narcolepsy. The results of these studies confirm that GHB treatment substantially reduces the signs and symptoms of narcolepsy (e.g., daytime sleepiness, cataplexy, sleep paralysis, apnea, and hypnagogic hallucinations). In addition, GHB increases total sleep time and REM sleep, and decreases REM latency. Results of these studies show a positive safety profile for GHB when used long-term for the treatment of narcolepsy. Adverse experiences with GHB have been minimal in incidence and degree of severity and include episodes of sleepwalking, enuresis, headache, and dizziness.

GHB and γ-butyrolactone are available from the Aldrich Chemical Co., Milwaukee, Wis., and can be employed to prepare other compounds within the scope of formula (I). The compound can be esterified with (C₁-C₄)alkanols and alkanoylated or benzoylated with alkanoyl and benzoyl chloride or anhydrides. The cation can also be readily exchanged to replace sodium with other metal or organic cations, such as Ca⁺, K⁺, Li⁺, or (R)₄ N⁺ wherein each R is H, phenyl, (C₁-C₆)alkyl or hydroxy(C₁-C₆)alkyl, i.e., ammonium or hydroxyethyl amine salts. For preparation methods for 4-hydroxy-butanoic acid and its derivatives, see, Marvel et al., J. Am. Chem. Soc., 51:260 (1929); Japanese Patent No. 63174947, and German Patent Nos. 237310, 237308 and 237309.

While the mechanisms of GHB's anti-hyperkinetic effects are unknown, and without wishing to be bound to a particular theory, the drug likely acts to change the metabolic topography of cortical-subcortical circuits responsible for generating myoclonic movements. In a recent study, it was shown that patients with posthypoxic myoclonus have a specific pattern of hypermetabolism of the ventrolateral thalamus and pons. Frucht S. J., et al., Neurology 62:1879-1881 (2004). This area of the thalamus has been shown to be involved in other forms of myoclonus, and selected patients with myoclonus-dystonia have benefited from thalamic stimulation. Trottenberg T., et al., Mov. Disord. 16:769-771 (2001); Kupsch A., et al., J. Neurol. Neurosurg. Psychiatry 67:415-416 (1999).

The present invention is directed to methods for treating movement disorders such as hyperkinetic movement disorders by administering to a patient a compound of formula (I). Nonlimiting examples of movement disorders contemplated by the invention include myoclonus, dystonia, chorea, tics, and tremor, including essential tremor. The various types of myoclonus are categorized based on the physiology of the disease (cortical, subcortical, spinal, peripheral), clinical manifestations (anatomic distribution, provocative factors, contraction patterns) and also the cause of the myoclonic movements (physiologic, essential myoclonus, myoclonic epilepsy, secondary myoclonus). For example, cortical myoclonus arises from the sensorimotor cortex and is typified by the regular rhythms of their jerking movements. Subcortical myoclonus arises from damage to the thalamus or brainstem. Myoclonus can be focal (affecting a specific body part), segmental (affecting parts of the body that are near each other), multifocal or generalized. Some myoclonic movements are spontaneous while others occur in response to stimulus (reflex or stimulus-sensitive). Action or intention myoclonus occurs during voluntary movements or the intention to move. The contraction patterns of myoclonic movement can be rhythmic, arrhythmic, or oscillatory. In one embodiment, the myoclonus is not alcohol sensitive essential myoclonus-dystonia. In other embodiments, the myoclonus is posthypoxic myoclonus or not alcohol responsive posthypoxic myoclonus. In further embodiments, the patient exhibits one or more of the following: negative myoclonus, myoclonus at rest, stimulus-sensitive myoclonus and action myoclonus. In yet further embodiment, the myoclonus is palatal myoclonus, a startle syndrome, spinal myoclonus, progressive myoclonic epilepsy, and brainstem myoclonus.

Tremor also encompasses a variety of types. Kinetic or intention tremor occurs during voluntary movement. Postural tremor, one type of kinetic tremor, occurs when attempting to maintain a fixed position against gravity, such as outstretched arms. Internal tremor is a general vibrating sensation. Typically, tremors disappear during sleep. Tremor is evaluated by determining the distribution of the tremor, the type and how it occurs, intensity and frequency, contraction pattern and degree of functional performance. In some embodiments, the patient exhibits one or more of the following: enhanced physiologic tremor, postural tremor, or kinetic tremor.

The invention is also directed to treatment of dystonia, cerebellar tremor, a tic or chorea with compounds of formula (I). The various types of dystonia are characterized based on anatomical distribution. For example, focal dystonia is limited to one area of the body, whereas segmental and multifocal dystonias affect two or more areas of the body (nearby/contiguous and more distant, respectively). Hemidystonia affects one half of the body, while generalized dystonia involves leg movement in additional to one or more other regions of the body. Examples of focal dystonia include, without limitation, cervical dystonia, blepharospasm, oromandibular dystonia, laryngeal dystonia, and limb dystonia. With primary, or idiopathic, dystonia, the dystonia is present without other neurologic abnormalities, and secondary causes are ruled out, with the exception of tremor. Other classifications for dystonia include secondary (sympathetic) dystonia, dystonia-plus syndromes (e.g., dystonia with myoclonus), and heredodegenerative dystonia. The invention is further directed to focal-task dystonia.

Some non-limiting types of movement disorders that the invention is directed to treating are chorea, ballismus, and tics. Chorea is an involuntary, non-rhythmical movement of moderate speed and amplitude that passes quickly and randomly from one part of the body to the other. It may involve the face and limbs, and can result in the inability for a patient to maintain posture. Chorea occurs in patients with drug-induced disorders or hereditary diseases, such as Huntington's disease. Ballismus consists of irregular and unpredictable movements that can be high in amplitude and velocity. In most subjects, ballismus is limited to one side of the body and can be caused by a focal destructive lesion of the contralateral subthalamic nucleus. It is often referred to as hemiballismus. Tics comprise abnormal, repetitive movements or sounds, which are subsequently classified as motor tics and vocal tics, respectively. These impulsive actions are random and variable in pattern. Non-limiting examples of tics include facial movement, repetitive eye blinks, head shakes, and vocalizations. Tourette's syndrome is the best-known condition characterized by vocal and motor tics.

In some embodiments, the subject is a mammal. Nonlimiting examples of mammals include: human, primate, mouse, otter, rat, and dog.

The invention also provides methods for ameliorating one or more movement disorders by administering to a patient a compound of formula (I). In one embodiment, the invention provides methods for ameliorating one or more myoclonic movements by administering to a patient a compound of formula (I). Nonlimiting examples of myoclonic movements include: negative myoclonus, myoclonus at rest, stimulus-sensitive myoclonus, action myoclonus, alcohol responsive posthypoxic myoclonus, palatal myoclonus, a startle syndrome, and spinal myoclonus. In one nonlimiting embodiment, the functional performance of a patient diagnosed with myoclonus is improved by administering to the patient a compound of formula (I). In one embodiment, the amelioration of myoclonic movements and the improvement of functional performance are assessed by use of the Unified Myoclonus Rating Scale. In another embodiment, the amelioration of myoclonic movements and the improvement of functional performance are assessed by use of the Chadwick-Marsden Scale.

Amelioration of one or more tremors is achieved by administering to a patient a compound of formula (I). Examples of tremors ameliorated by administration of a compound of formula (I) include without limitation: hand tremor, arm tremor, voice tremor, head tremor, trunk tremor and/or leg tremor. Amelioration is assessed by use of tremor classification scales known to persons skilled in the art, such as the Collaborative Clinical Classification of Tremor, the Classification of Essential Tremor (Tremor Research Investigation Group) or the WHIGET (Washington Heights Inwood Genetic Essential Tremor) Rating Scale.

Amelioration of dystonia, cerebellar tremor, a tic or chorea is achieved by administrating to a patient a compound of formula (I). Examples of dystonias ameliorated by administration of a compound of formula (I) include generalized dystonias, focal dystonias, action dystonia, task-specific dystonia, rest dystonia, segmental dystonias, multifocal dystonias, hemidystonia, cervical dystonia, blepharospasm, oromandibular dystonia, laryngeal dystonia, and limb dystonia. Amelioration of dystonia, cerebellar tremor, a tic or chorea is assessed by methods known to those of skill in the art.

Administration and Dosages

While it is possible that, for use in therapy, the compounds of formula (I), such as 4-hydroxybutyric acid salts, may be administered as the pure chemicals, as by inhalation of a fine powder via an insufflator, it is preferable to present the active ingredient as a pharmaceutical formulation. The invention, thus, further provides a pharmaceutical formulation comprising a compound of formula (I), together with one or more pharmaceutically acceptable carriers therefor and, optionally, other therapeutic and/or prophylactic ingredients. The cations and carrier(s) must be “acceptable” in the sense of being compatible with the other ingredients of the formulation and not deleterious to the recipient thereof.

Pharmaceutical formulations include those suitable for oral or parenteral (including intramuscular, subcutaneous and intravenous) administration. Forms suitable for parenteral administration also include forms suitable for administration by inhalation or insufflation or for nasal, or topical (including buccal, rectal, vaginal and sublingual) administration. The formulations may, where appropriate, be conveniently presented in discrete unit dosage forms and may be prepared by any of the methods well known in the art of pharmacy. Such methods include the step of bringing into association the active compound with liquid carriers, solid matrices, semi-solid carriers, finely divided solid carriers or combinations thereof, and then, if necessary, shaping the product into the desired delivery system.

Pharmaceutical formulations suitable for oral administration may be presented as discrete unit dosage forms such as hard or soft gelatin capsules, cachets or tables each containing a predetermined amount of the active ingredient; as a powder or as granules; as a solution, a suspension or as an emulsion; or in a chewable base such as a synthetic resin or chicle for ingestion of the active ingredient from a chewing gum. The active ingredient may also be presented as a bolus, electuary or paste. Tablets and capsules for oral administration may contain conventional excipients such as binding agents, fillers, lubricants, disintegrants, or wetting agents. The tablets may be coated according to methods well known in the art, i.e., with enteric coatings.

Oral liquid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, flavoring, emulsifying agents, non-aqueous vehicles (which may include edible oils), or preservatives.

The compounds according to the invention may also be formulated for parenteral administration (e.g., by injection, for example, bolus injection or continuous infusion) and may be presented in unit dose form in ampules, pre-filled syringes, small volume infusion containers or in multi-dose containers with an added preservative. The compositions may take such forms as suspensions, solutions, or emulsions in oily or aqueous vehicles, and may contain formulatory agents such as suspending, stabilizing and/or dispersing agents. Alternatively, the active ingredient may be in powder form, obtained by aseptic isolation of sterile solid or by lyophilization from solution, for constitution with a suitable vehicle, e.g., sterile, pyrogen-free water, before use.

For topical administration to the epidermis, compound(s) of formula (I) may be formulated as ointments, creams or lotions, or as the active ingredient of a transdermal patch. Suitable transdermal delivery systems are disclosed, for example, in A. Fisher et al., (U.S. Pat. No. 4,788,603), Chien et al., (U.S. Pat. No. 5,145,682) or R. Bawa et al., (U.S. Pat. Nos. 4,931,279, 4,668,506 and 4,713,224). Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents. The active ingredient can also be delivered via iontophoresis, e.g., as disclosed in U.S. Pat. Nos. 4,140,122, 4,383,529, or 4,051,842.

Formulations suitable for topical administration in the mouth include unit dosage forms such as lozenges comprising active ingredient in a flavored base, usually sucrose and acacia or tragacanth; pastilles comprising the active ingredient in an inert base such as gelatin and glycerin or sucrose and acacia; mucoadherent gels, and mouthwashes comprising the active ingredient in a suitable liquid carrier.

When desired, the above-described formulations can be adapted to give sustained release of the active ingredient employed, e.g., by combination with certain hydrophilic polymer matrices, e.g., comprising natural gels, synthetic polymer gels or mixtures thereof.

Pharmaceutical formulations suitable for rectal administration wherein the carrier is a solid are most preferably presented as unit dose suppositories. Suitable carriers include cocoa butter and other materials commonly used in the art, and the suppositories may be conveniently formed by admixture of the active compound with the softened or melted carrier(s) followed by chilling and shaping in molds.

Formulations suitable for vaginal administration may be presented as pessaries, tampons, creams, gels, pastes, foams or sprays containing, in addition to the active ingredient, such carriers as are known in the art to be appropriate.

For administration by inhalation, the compounds according to the invention are conveniently delivered form an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

Alternatively, for administration by inhalation or insufflation, the compounds according to the invention may take the form of a dry powder composition, for example, a powder mix of the compound and a suitable powder base such as lactose or starch. The powder composition may be presented in unit dosage form in, for example, capsules or cartridges or, e.g., gelatin or blister packs from which the powder may be administered with the aid of an inhalator or insufflator.

For intra-nasal administration, the compounds of the invention may be administered via a liquid spray, such as via a plastic bottle atomizer. Typical of these are the Mistometer® (Winthrop) and the Medihaler® (Riker).

The pharmaceutical compositions according to the invention may also contain other adjuvants such as flavorings, colorings, antimicrobial agents, or preservatives.

The methods for treating movement disorders according to the invention may also include co-administration of a compound of formula (I) with one or more anti-myoclonic, anti-tremor, anti-chorea, anti-tic, or anti-dystonia agents. In one embodiment, the compounds are co-administered simultaneously. In another embodiment, the compounds are co-administered sequentially. Anti-myoclonic and anti-tremor agents are well known to those of skill in the art. For example, benzodiazepines, anticonvulsants, and β-adrenergic blockers are known to be effective in treating myoclonus and essential tremor and are suitable for co-administration with GHB. In addition, GABA receptor agonists are suitable for co-administration with GHB. Examples of anti-myoclonic agents include, without limitation, clonazepam, levetiracetam, valproic acid, phenobarbital, topiramate, zonisamide, primidone, phenyloin, 5-hydroxytryptophan, piracetam, acetazolamide, baclofen, fluoxetine, propranolol, lamotrigine, sumatriptan, tetrabenazine, trihexyphenidyl, melatonin, and alprazolam. Examples of anti-tremor agents include, without limitation, mysoline, propranolol, primidone, benzodiazepines (clonazepam, lorazepam, alprazolam, diazepam) nadolol, methazolamide, gabapentin, topiramate, levetiracetam and botulinum toxin. Some non-limiting examples of anti-chorea medications include haloperidol, reserpine, tetrabenazine, and valproic acid. Anti-tic medications include, but are not limited to clonidine, clonazepam, guanfacine, haloperidol, pimozide, and tetrabenazine. It will be appreciated that the compound of formula (I) and the co-administered agent can be prepared in a single pharmaceutical composition or can be administered as separate pharmaceutical compositions. Examples of anti-dystonia agents include, without limitation, dopaminergic drugs (e.g., dopamine agonists, dopamine-blocking agents, dopamine-depleting agents, tetrabenasize with or without lithium, clozapine, olanzapine), botulinum toxin, and benzodiazepines (diazepam, clonazepam, lorazepam, alprazolam,), baclofen, and anticholinergics (trihexyphenidyl, diphenhydramine).

It will be further appreciated that the amount of the compound of formula (I) required for use in treatment will vary not only with the particular compound selected but also with the route of administration, the severity of the condition being treated and the age and condition of the patient and will be ultimately at the discretion of the attendant physician or clinician.

In general, however, a suitable dose will be in the range of that shown to be effective as a hypnotic agent, i.e., to treat narcolepsy, of from about 1-500 mg/kg, e.g., from about 10-250 mg/kg of body weight per day, such as 25 to about 200 mg per kilogram body weight of the recipient per day. In one embodiment, a total daily dosage of 500 mg-20 g is administered. In another embodiment, a total daily dosage of 2-12 g is administered. In other embodiments, the total daily dosage is about 4.5 g. In still further embodiments, the total daily dosage is 4, 6, or 8 g. In some embodiments, the compound of formula (I) is administered twice daily. In other embodiments, the compound of formula (I) is administered three times daily.

The compound is conveniently administered in unit dosage form; for example, containing 0.5-20 g, conveniently 1-7.5 g, most conveniently, 2-5 g, of active ingredient per unit dosage form.

The total daily dosage, i.e., of about 500 mg-20 g is administered three or more times daily for about 1-4 months or longer, as needed. In other embodiments, the total daily dosage is administered chronically, i.e., with no time limit for ending the therapy.

The desired dose may conveniently be presented in a single dose or as divided doses administered at appropriate intervals, for example, as two, three, four or more doses or sub-doses per day. The sub-dose itself may be further divided, e.g., into a number of discrete loosely spaced administrations such as teaspoons of a liquid composition or multiple inhalations from an insufflator. In one embodiment, a dose of 1-4 g is administered twice daily.

In some embodiments, in order to guard against a possible increased sensitivity to the sedative effects of Xyrem®, the subject receives a dose of 2 grams per night. After a period of time to adjust to the medication (e.g., 2 weeks), an assessment is performed to determine whether or not the myoclonus is still present and troublesome. If so, the dose is increased to 4 grams per night. After another period of time to adjust to the medication, (e.g., another 2 weeks), a similar assessment is made, and if needed the dose is increased to 6 grams per night. The dose is further increased in similar fashion as needed.

When the compound of formula (I) is administered once daily, the compound is administered in the morning, afternoon, evening or night before bedtime. Because hyperkinetic movement disorders disappear during sleep, administration during the day allows for observation of the effects of the drug. When administered twice daily, the first dose is administered in the morning or, alternatively, in the afternoon. The second dose is administered approximately 6-12 hours later. For example, in the afternoon, evening or nighttime before bed.

The following examples illustrate the present invention, and are set forth to aid in the understanding of the invention, and should not be construed to limit in any way the scope of the invention as defined in the claims which follow thereafter.

### EXAMPLES

### Example 1

A single patient clinical trial of Xyrem® for severe posthypoxic myoclonus was performed. The patient was a 37-year-old woman who suffered an anesthesia accident. After remaining in a coma, she awakened and gradually recovered, however she was completely disabled by severe myoclonic jerks that affected her voice, head, proximal arms, legs and trunk. By clinical examination she had both positive myoclonus (active jerks) and negative myoclonus (postural lapses). Her myoclonus had been treated with phenobarbital, zonisamide, clonazepam and levetiracetam, without significant improvement. Prior to the trial she was treated with clonazepam and levetiracetam. She is allergic to penicillin, has no other known drug allergies, and is otherwise in good general health.

Case Report

An open-label, dose-finding, blinded rating trial of GHB in a single patient with severe, debilitating alcohol-responsive posthypoxic myoclonus refractory to treatment with standard anti-myoclonic agents was conduced. GHB was given in divided doses during the day and was well tolerated. Intensity and severity of myoclonus was measured using the Unified Myoclonus Rating Scale (UMRS), a non-invasive clinical rating scale. In addition, the patient was videotaped while the UMRS was performed, so that myoclonus could be scored by a blinded rater. These methodologies demonstrated complete resolution of myoclonus at rest and stimulus-sensitive myoclonus. Action myoclonus and functional performance also improved in ways that were practically meaningful, allowing the patient to feed herself, to accomplish daily hygiene tasks, and to walk with assistance.

The patient was referred to the Columbia University Medical Center Movement Disorder Center for evaluation of severe posthypoxic myoclonus at the age of 37. At the age of 34, she underwent an elective uterine fibroid myomectomy that was complicated by an anesthesia accident. The duration of hypoxia or cardiac arrest was unknown; however, frequent tonic-clonic seizures were noted immediately in the post-operative period. She remained comatose and intubated, and subsequently required a tracheotomy and feeding tube. Myoclonic jerks and electrographic seizures were noted in the intensive care unit. She developed severe, debilitating myoclonus following the anesthesia accident. Her mental status and cognition were completely normal, but she was totally dependent and wheelchair-bound due to severe, incapacitating myoclonus, which resisted treatment with all standard myoclonus drugs. After a lengthy hospitalization, she was transferred to a rehabilitation center and finally returned home three years after the event in a wheelchair-bound, fully dependent state due to severe myoclonus. Multiple trials of anti-myoclonic medications including clonazepam, valproic acid, phenobarbital, topiramate, zonisamide and levetiracetam were only minimally successful. She could not tolerate valproic acid secondary to a drop in platelet count, and zonisamide produced anorexia. Of these agents, only clonazepam partially improved the severity of her myoclonus, with dose-limiting sedation. An MRI of the brain revealed mild atrophy and no evidence of ischemic or structural injury. Back-averaged EEGs and somatosensory evoked potentials were not available.

Anti-myoclonic medications on initial evaluation included clonazepam (3 mg daily) and levetiracetam (2,500 mg daily). Neurologic examination revealed a thin cooperative woman with obvious severe myoclonic jerks. Detailed mental status examination was hampered by severe myoclonic speech; however, she answered all questions appropriately and followed complex commands without difficulty. By her family's report, her short-term and long-term memory were not impaired. At rest, positive myoclonic jerks of the arms, legs and trunk were evident. These jerking motions were exacerbated when the patient attempted voluntary movement, affecting in particular the proximal area of the arms. Negative myoclonic jerks were frequent, affecting the arms and wrists in outstretched posture. Exaggerated startle to sound and threat was also present. Stimulus-sensitivity to pin and reflex were difficult to assess due to the frequency of resting myoclonus. Myoclonus of the arms prevented her from performing finger to nose testing, holding objects such as a cup, spoon or pen, or even maintaining her arms in an outstretched posture. She was unable to arise from a chair, and, on standing, severe negative myoclonic jerks of muscles of postural support prevented ambulation.

Methods

Given the lack of significant benefit from treatment with standard anti-myoclonic agents, it was felt that empiric trials of other anti-epileptic agents were unlikely to improve her condition. She had not received piracetam; however, based on her lack of response to levetiracetam, the difficulty in obtaining piracetam, and her examination (suggesting a prominent subcortical component of myoclonus), it was not expected that she would respond well with this agent. After obtaining verbal consent from the patient and her husband, she was given approximately six ounces of wine to drink (alcohol content 14% by volume). Within 30 minutes, dramatic improvement in myoclonus was obvious to the patient and her family. She was able to speak, to use her hands, to feed herself, and even walk with mild assistance. She was videotaped before and after the trial with alcohol. Resting myoclonic jerks resolved, her speech returned to near normal, and she was able to use her arms in fluid gestures for the first time in three years. Mild positive and negative myoclonic jerks were still present on forward arm extension, although both were improved. She was able to stand with assistance and even walk with gentle guidance. She appeared slightly euphoric from the alcohol, but was not sedated. The anti-myoclonic effect of alcohol resolved in several hours.

Given the dramatic response to alcohol, a single-patient, open-label, dose-ranging trial to assess the tolerability and efficacy of GHB (Xyrem®, Orphan Medical, Inc.) as a treatment for alcohol-responsive posthypoxic myoclonus was designed, which was approved by The Columbia University Medical Center Institutional Review Board. After obtaining informed consent, she was videotaped during performance of the Unified Myoclonus Rating Scale (UMRS). The UMRS is a validated clinical rating instrument that measures the severity and intensity of myoclonus and has been used in other trials of anti-myoclonic agents. Frucht S. J., et al., Adv Neurology Vol. 89 Lippincott Williams and Wilkins, Philadelphia, 2002:361-376. The scale consists of eight sections: section I: patient questionnaire (11 items); section II: myoclonus at rest (frequency and amplitude, 16 items); section III: stimulus-sensitive myoclonus (17 items); section IV: severity of myoclonus with action (frequency and amplitude, 20 items); section V: performance on functional tests (5 items); section VI: physician rating of patient's global disability (1 item); section VII: presence of negative myoclonus (1 item); section VIII: severity of negative myoclonus (1 item). Each item is rated on a scale of 0 to 4, with higher scores assigned to myoclonus of greater severity or frequency. For section III stimulus-sensitivity is either present (1) or absent (0); section VII negative myoclonus either present (1) or absent (0); and section VIII negative myoclonus absent (0) to severe (3).

All office visits and the UMRS scale were performed by the supervisory physician. The patient came to the office for five visits, each separated by two weeks (time 0, 2 weeks, 4 weeks, 6 weeks and 8 weeks). At each visit, the physician examined the patient, and inquired about any adverse events. The physician performed the UMRS while the patient was videotaped. Xyrem® was dispensed by the physician at each visit.

At the initial visit the UMRS was performed and videotaped, 1 gm of GHB was administered by mouth, and she was monitored in the office for one hour. She then took 1 gm twice daily for two weeks, at which time she returned to the office. One hour after receiving 2 grams of GHB, the UMRS was performed and videotaped and for the next two weeks she took 2 grams twice daily. Two weeks later, the UMRS was performed and videotaped one hour after receiving 3 grams of GHB, and after two weeks of 3 grams twice daily, the same procedure was performed after receiving 4 grams in the office. After taking 4 grams twice daily for two weeks, she returned for UMRS videotaping, and the decision was made to continue the target dose of 4 gm twice daily.

A movement disorder neurologist blinded to the trial design scored videotapes of the baseline visit, 2 gm, 3 gm, 4 gm (first) and 4 gm (second) visits in random order. The blinded rater was not provided with treatment scores of section I of the UMRS (patient self-assessment) in order to maintain the rating blind. Scores for each section were calculated as described previously. Frucht S. J., et al., Adv Neurology Vol. 89. Lippincott Williams and Wilkins, Philadelphia, 2002:361-376.

Results

The patient reported slight dizziness after ingesting the first 1 gm dose of GHB, and also commented that the oral solution tasted salty. Little difference was seen in the patent's clinical examination after the initial 1 gm dose, and due to patient fatigue the UMRS was not repeated. Within 20 minutes of administering the first 2 gm dose, marked diminution of myoclonus at rest and stimulus-sensitivity, and moderate improvement of myoclonus with action was obvious to the patient, her family and the examining physician. This benefit peaked at one hour and lasted approximately 3 hours. Improvement in myoclonus was also noted when she received the 3 gm and 4 gm doses in the office, with a dose-dependent effect. She tolerated the increasing doses without significant sedation or adverse events. Both positive and negative myoclonic jerks appeared to improve with treatment. She regained the ability to hold a cup and use utensils such as chopsticks, to write (albeit very slowly), and to walk with assistance.

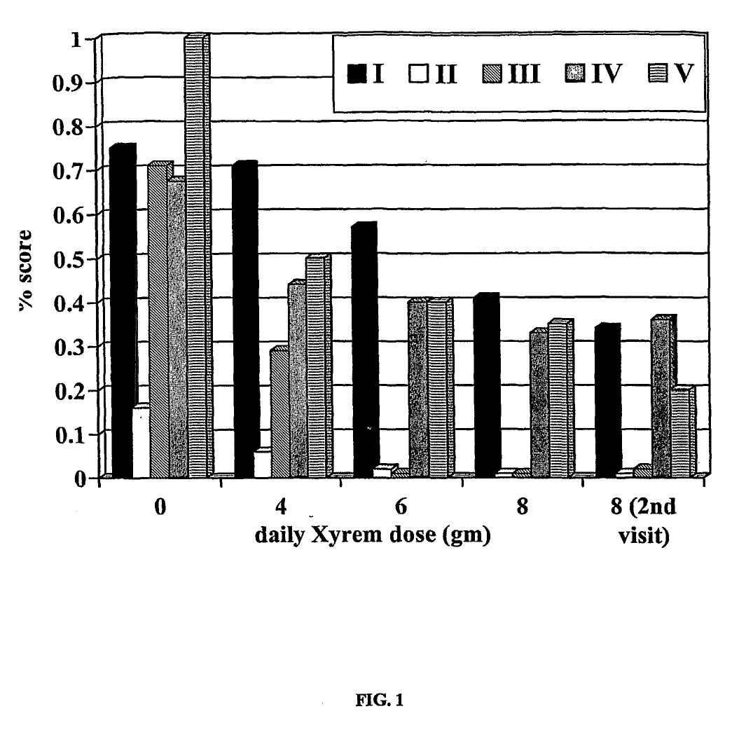

Scores of each subsection of the UMRS are presented in Table 1 and plotted in FIG. 1. The scores reflect the intensity and the frequency of the myoclonic movement. Lower scores indicate a lower occurrence and intensity of the jerking movement. The data presented in FIG. 1 demonstrates the % of the maximum score for each UMRS subsection at each dosage level. For example, at a dosage level of 0 gm/day, the score of subsection II was 3, which is 75% of the maximum score of 4. As demonstrated in Table 1 and FIG. 1, myoclonus at rest (section II) dramatically diminished after 2 gm of GHB and disappeared at higher doses. Stimulus-sensitive myoclonus (section III) improved comparably. Action myoclonus (section IV) improved in dose-dependent fashion, although there was residual impairment on this subsection. Functional performance in writing, spiral copying, pouring and using a spoon (section V) improved by 80%. Blinded videotape review did not confirm the examiner's impression that negative myoclonus improved with treatment.

TABLE 1
UMRS SUBSCORES BEFORE
AND DURING TREATMENT WITH GHB

| | Max. | | | | | 4 gm |
|---|---|---|---|---|---|---|
| | UMRS | | | | | (2nd |
| UMRS Subsection | score | 0 gm | 2 gm | 3 gm | 4 gm | visit) |
| I (Self Assessment) | 44 | 33 | 31 | 25 | 18 | 15 |
| I (Global Disability) | 4 | 3 | 3 | 2 | 2 | 2 |
| II (Myoclonus @ Rest) | 128 | 21 | 8 | 3 | 1 | 1 |
| III (Stimulus-Sens.) | 17 | 12 | 5 | 1 | 1 | 3 |
| IV (Action Myoclonus) | 160 | 108 | 70 | 64 | 53 | 58 |
| V (Funct. Performance) | 20 | 20 | 10 | 8 | 7 | 4 |
| VI (MD Global Disab.) | 4 | 3 | 2 | 2 | 2 | 2 |
| VII (Neg. Myoclonus) | 1 | 1 | 1 | 1 | 1 | 1 |
| VIII (Severity) | 3 | 2 | 2 | 2 | 2 | 2 |

In this open-label, single-patient trial, oral GHB was markedly effective in ameliorating severe alcohol-sensitive posthypoxic myoclonus. Despite the possibility of placebo benefit due to the open-label design, it is unlikely that the patient's desire to improve substantially affected her myoclonus scores. Each dose of GHB greater than or equal to 2 gm worked in a highly reproducible fashion, exerting its peak effect in one hour and wearing off in 3½ hours. Benefits were clearly dose-dependent, as measured both by her self-ratings of disability (section I of the UMRS), and blinded ratings of sections II-V. The blinded rating of myoclonus scores makes it unlikely that investigator bias significantly affected the results.

The clinical trial was designed with a very slow titration schedule, in order to prevent possible side effects such as sedation, worsening of ataxia or orthostasis. Like most forms of myoclonus, posthypoxic myoclonus disappears during sleep and it is therefore necessary to administer the drug during waking hours in order to observe its effects. The patient was able to tolerate two daytime doses of 4 gm of GHB without significant sedation, even with concurrent treatment with clonazepam and levetiracetam.

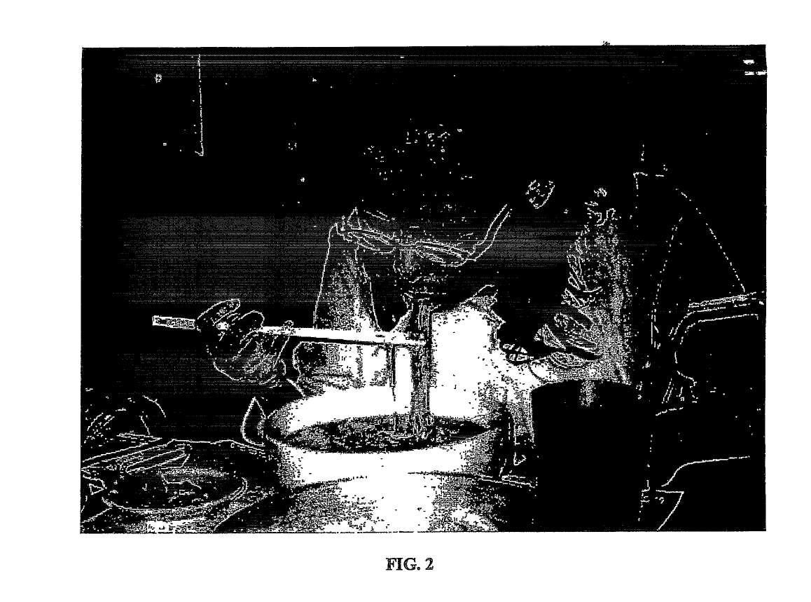

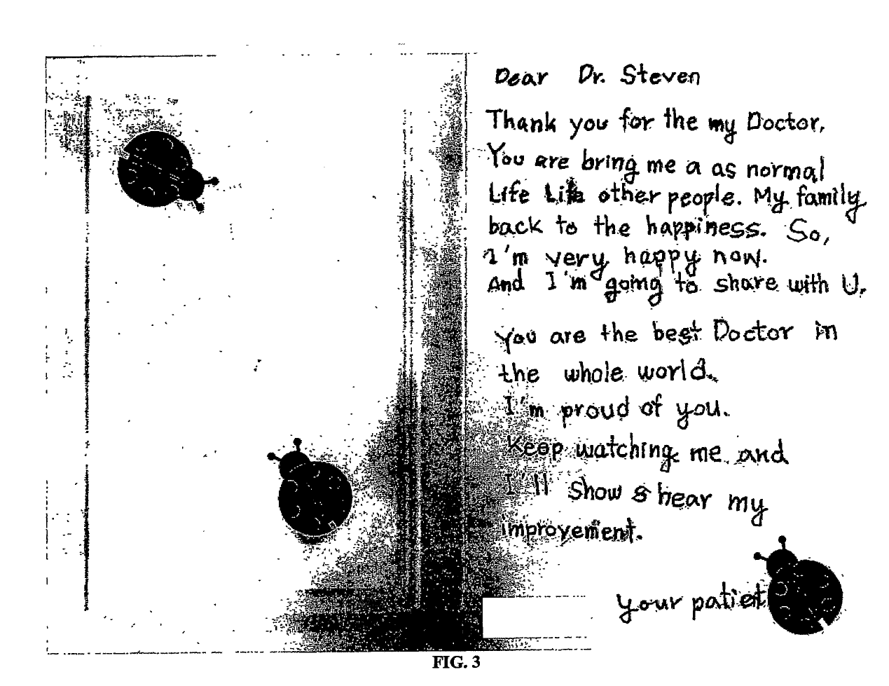

The most dramatic improvements in myoclonus scores were observed in sections II (myoclonus at rest) and III (stimulus-sensitivity) of the UMRS. Both resting and stimulus-sensitive myoclonus markedly improved at doses of 2 gm twice daily, and both virtually disappeared at 3 gm twice daily. Action myoclonus also improved in a dose-dependent fashion, but residual deficits remained at the 4 gm twice-daily dose. The action myoclonus subscore measures both the frequency and amplitude of myoclonus triggered by movements of the face, neck, trunk, arms and legs, as well as myoclonus triggered by arising, standing and walking. While these subsection scores may reflect a less robust anti-myoclonic effect on action myoclonus, limb ataxia may inadvertently contribute to the scores of arm and leg movements on part IV of the scale. For example, a breakdown in fluency of movement on finger to nose or heel to shin testing may be attributed to myoclonus, when in fact the deficits arise from underlying cerebellar dysfunction. Many patients with posthypoxic myoclonus have mild cerebellar deficits (Agarwal P., et al., Curr. Opin. Neurol. 16:515-521 (2003)), and section IV of the UMRS is not designed to parse out these components. In contrast, improvements in functional performance observed in section V were more dramatic. These tests measure tasks that patients must perform to assume responsibility for their care, such as writing, pouring and using utensils to eat or drink. After treatment with Xyrem® the patient's motor control improved to the point of being able to feed herself with chopsticks (FIG. 2). No time limitations are imposed so that the patient's best possible performance is measured. Although the patient required several minutes to perform the writing samples and more than one hour to complete a written passage of a six sentences (FIG. 3), these tasks were completely impossible prior to treatment with GHB. After treatment with GHB, the patient was able to write clearly, albeit at a slow pace.

It was surprising to find that blinded videotape review did not confirm the impression of the examiner and the patient's family that negative myoclonic jerks of the limbs and torso improved with alcohol and GHB. It was the examiner's impression that improvement in walking was related to better control of postural negative myoclonic jerks. Negative myoclonus is typically seen in the setting of metabolic derangements or toxin exposures. Agarwal P., et al., Curr. Opin. Neurol. 16:515-521 (2003). It may also occur as a rare reflex-induced phenomenon in patients with cortical reflex negative myoclonus. Shibasaki H., et al., Brain 117:477-486 (1994). Aside from the latter setting where treatment with anti-epileptics may be helpful, negative myoclonus is notoriously refractory to treatment. It is therefore possible that GHB may be a useful agent to treat negative myoclonus.

### Example 2

In this prophetic example, a short double-blind, placebo-controlled protocol will be performed with approximately 20 patients, followed by an open-label extension. The protocol will call for a titration up to 6.125 gm per day in the double-blind phase, with the option of titrating up to 9 gm per day in the open-label phase.

Experimental Design

The study is a double-blind, randomized, placebo-controlled, parallel-group, dose ranging trial of GHB for dystonia. The study population includes patients with clinically significant myoclonus-dystonia.

The primary objectives include: 1) To assess the safety and tolerability of GHB in dystonia patients and 2) To assess the efficacy of GHB in treating dystonia. The secondary objectives are: 1) To assess the effect of dosing of GHB on dystonia.

The duration of the double-blind portion of the study is 8 weeks. The duration of the fixed-dose portion of the study is 8 weeks. The duration of the dose-ranging portion of the study would be an extended period, perhaps as long as one year.

In order to qualify for this study, patients must meet the following criteria: 1) Men and women, diagnosed with dystonia. The myoclonus must be present for at least one year by history. 2) Age≧18. 3). Patients may be treated with other medications for myoclonus, including clonazepam, valproic acid, and phenobarbital. All medications must remain stable for a period of 4 weeks prior to screening. 4) Women of childbearing age must not be pregnant, and must use adequate birth control for the duration of the study. 5) Patients must have clinically significant dystonia. 6) Patients must be able and willing to comply with the study visits and procedures. 7) Patients must be able to give informed consent.

The following patients are excluded from the study: 1) Patients with a clinically significant medical condition, including hepatic or renal disease. 2) Patients with a MMSE score of ≧24. 3) Patients with a history of a clinically significant psychiatric illness, including major depression and psychosis. 4) Patients unwilling to abstain from alcohol for the duration of the study. 5) Patients with a history of substance abuse. 6) Patients who do not demonstrate willingness and ability to comply with all aspects of the protocol, including drug accountability.

The primary outcome measures of the study are the change in the UMRS. The UMRS is a statistically validated comprehensive clinical rating tool for evaluating patients with myoclonus.

Data Analysis

Videotapes of the UMRS are performed at each patient visit. The videotapes and UMRS forms are collected, and rated by two raters who are blinded to patients' treatment status. Ratings will be entered into a database, and analyzed by a biostatistician. The following assessments are performed periodically throughout the study: medical and neurological history, physical exam, vital signs, laboratory tests, pregnancy test, lead, EKG, UMRS, MMSE, depression inventory, adverse events, concomitant therapy, drug compliance.

All UMRS examinations are videotaped, and observers who are blinded to patient's treatment status score the videotapes.

Visits in the open-label dose-ranging phase will occur at three months, six months and one year. Assessments at these visits will include those performed at the screening visit.

### Example 3

### Patients and Methods

Five patients were enrolled in a trial from the Movement Disorders Division of Columbia University Medical Center during the fall of 2004. All patients were afflicted with hyperkinetic movement disorders that responded to ethanol (defined as a noticeable change to the patient), and all were refractory to treatment with conventional medications or could not tolerate them. The Medical Center's Institutional Review Board approved the trial, and written and verbal informed consent were obtained from all patients prior to enrollment. Salient clinical features appear below and are summarized in Table 1.

TABLE 1
Clinical features of patients with ethanol-responsive movement disorders

| Pt # | M/F | Age | Dx | t (yrs) | CURRENT RX | Past Rx |
|---|---|---|---|---|---|---|
| 1 | F | 37 | PHM | 6 | levetiracetam 2,500 mg | valproic acid |
| | | | | | clonazepam 6 mg | tizanidine |
| | | | | | phenobarbital 60 mg | primidone |
| | | | | | alprazolam 0.5 mg | gabapentin |
| | | | | | rabeprazole 20 mg | paroxetine |
| | | | | | baclofen 20 mg | piracetam |
| 2 | M | 25 | MD | 7 | levetiracetam 500 mg | trihexiphenidyl |
| | | | | | diazepam 15 mg | clonazepam |
| | | | | | | primidone |
| 3 | M | 20 | MD | 17.5 | paroxitene 20 mg | trihexiphenidyl |
| | | | | | clonazepam 2 mg | L-5-hydroxytryptophan |
| | | | | | | valproic acid |
| | | | | | | baclofen |
| | | | | | | levetiracetam |
| 4 | M | 67 | ET | 50 | none | propranolol |
| 5 | M | 75 | ET | 13 | levetiracetam 1,000 mg | primidone |

t—duration of symptoms in years on enrollment in the trial;
Dx—diagnosis;
Current Rx—current medications during the trial;
Past Rx—past medication exposures;
PHM—posthypoxic myoclonus;
MD—myoclonus-dystonia;
ET—essential tremor

Patient 1: A 37-year-old woman with a history of asthma suffered a cardiopulmonary arrest after a drug overdose at age 31, emerging from coma with severe PHM. On initial evaluation at our center at age 33, action and intention myoclonus were severe, with prominent vocal myoclonus and disabling negative myoclonic jerks of the trunk and legs. Her mother noted that ingestion of two glasses of wine noticeably improved her myoclonus, allowing her to assist in daily hygiene activities. Nine months prior to enrollment she sustained a subcortical infarct during a hospitalization for pneumonia, leaving her with a residual left hemiparesis.

Patient 2: A 25-year-old man presented to our medical center for evaluation of a seven-year history of myoclonic jerks. His family history was notable for a paternal grandmother with torticollis and two paternal first cousins with myoclonus, all ethanol-responsive; the patient however never consumed ethanol. Genetic testing revealed a mutation in the epsilon-sarcoglycan gene, confirming the diagnosis of MD. Klein C, et al. Am J Hum Genet 67:1314-9 (2000). Prominent proximal myoclonic jerks of the head, neck, and arms were triggered by voluntary actions such as pouring or writing.

Patient 3: A 20-year-old man presented at age 11 to our center for initial evaluation of myoclonus that began at age 2½ in his right foot. Myoclonic jerks of the trunk and proximal arms interfered with writing, pouring and using utensils. At age 17 he developed obsessive-compulsive symptoms that were successfully treated with paroxetine. Genetic testing revealed a mutation in the epsilon-sarcoglycan gene, confirming the diagnosis of MD. Klein C, et al. Am J Hum Genet. 67:1314-9 (2000) On several occasions he consumed ethanol, observing a dose-dependent improvement in myoclonus (requiring 80 gm of alcohol to reach maximal improvement).

Patient 4: A 67-year-old man with a family history of ET developed mild kinetic tremor of his hands in high school. Tremor progressively affected his ability to eat with utensils, hold a cup and write. His tremor was exquisitely alcohol-responsive with moderate tremor relief fifteen minutes after ingestion of one glass of wine, and near-complete tremor relief from two glasses. He chose not to take daily medication for his ET. Three years prior to enrollment he developed cervical dystonia that also responded to ethanol, and began receiving botulinum toxin injections. The last injection was performed seven weeks prior to enrollment.

Patient 5: A 75-year-old retired general surgeon developed a kinetic tremor of his hands at age 62, forcing him to retire. Action tremor of the hands became progressively severe, causing social embarrassment when eating in public. Because of severe chronic obstructive pulmonary disease, treatment with propranolol was contraindicated, and primidone was too sedating. He was not currently taking any medications for his pulmonary disease, which might worsen his tremor. He drank one or two glasses of wine on social occasions, with mild improvement in his tremor.

Clinical Trial Design

The dose and timing of all other drugs were kept constant throughout the trial, and patients were not withdrawn from other medications. Patients #1-3 were examined and videotaped using the Unified Myoclonus Rating Scale (UMRS), and patients #4 and 5 were examined and videotaped using the Washington Heights Inwood Tremor Rating Scale (WHIGET) (see Appendix I). Frucht S. J., et al. Adv Neurol 89:361-76 (2002); Louis, E. D., et al. Mov Disord 16:89-93 (2001).

APPENDIX I
MODIFIED UMRS SCORE SHEET
1. PATIENT QUESTIONNAIRE

| | |
|---|---|
| A. (Speech) | {0-4} |
| B. (Reading) | {0-4} |
| C. (Handwriting) | {0-4} |
| D. (Eating) | {0-4} |
| E. (Drinking) | {0-4} |
| F. (Swallowing) | {0-4} |
| G. (Hygiene) | {0-4} |
| H. (Dressing) | {0-4} |
| I. (Arising) | {0-4} |
| J. (Standing) | {0-4} |
| K. (Walking) | {0-4} |
| TOTAL | {0-44} |
| PT. GLOBAL ASSESSMENT | {0-4} |

2. MYOCLONUS AT REST

| Body | Frequency | | Amplitude | |
|---|---|---|---|---|
| Upper | | X | | {0-16} |
| Lower | | X | | {0-16} |
| Neck | | X | | {0-16} |
| Trunk | | X | | {0-16} |
| R Arm | | X | | {0-16} |
| L Arm | | X | | {0-16} |
| R Leg | | X | | {0-16} |
| L Leg | | X | | {0-16} |
| | | | TOTAL | {0-128} |

3. STIMULUS SENSTIVITY.
Score 1 if a stimulus produces a jerk in any body part; score 0 if no jerk
is elicited. Each stimulus is performed only once.

| Threat: thrust hands towards patient's face unexpectedly: | | |
|---|---|---|
| Claps hands unexpectedly: | | |
| Tap patient's nose with patient's eyes closed: | | |
| Elicit jaw jerk: | | |
| Pin prick: | On cheek | |
| | R arm (flexor surface of wrist) | |
| | L arm (flexor surface of wrist) | |
| | R leg (bottom of foot) | |
| | L leg (bottom of foot) | |
| Finger flick: | R index finger | |
| | L index finger | |
| Toe flick: | R great toe | |
| | L great toe | |
| Reflexes: | R bicep | |
| | L bicep | |
| | R knee jerk | |
| | L knee jerk | |
| | STIMULUS-SENSITIVITY TOTAL | {0-17} |

4. MYOCLONUS WITH ACTION

| Action | Frequency | | Amplitude | |
|---|---|---|---|---|
| A. Close Eyelids | | X | | {0-16} |
| B. Neck | | X | | {0-16} |
| C. Trunk | | X | | {0-16} |
| D. R arm | | X | | {0-16} |
| E. L arm | | X | | {0-16} |
| F. R leg | | X | | {0-16} |
| G. L leg | | X | | {0-16} |
| H. Arising | | X | | {0-16} |
| I. Standing | | X | | {0-16} |
| J. Walking | | X | | {0-16} |
| | | | TOTAL | {0-160} |

5. FUNCTIONAL TESTS

| | |
|---|---|
| A. Writing | {0-4} |
| B. R hand spiral | {0-4} |
| C. L hand spiral | {0-4} |
| D. R pouring | {0-4} |
| E. L pouring | {0-4} |
| F. R soup spoon | {0-4} |
| G. L soup spoon | {0-4} |
| TOTAL | {0-28} |

| | |
|---|---|
| 6. GLOBAL DISABILITY SCORE | {0-4} |

Sections 1-6 of the UMRS appear above. Full details regarding the scale are available in reference 16. Higher scores indicate more severe involuntary movements. The range for each section appears in brackets { }.

Side effects were defined as either minor or serious (leading to hospitalization) using good clinical practice standards (www.who.int/medicines/library/par/ggcp/GCPGuidePharmatrials), and patients were asked to report side effects at each visit. After initial examination and videotaping, patients were given 1 gm of sodium oxybate by mouth (2 ml of the standard 0.5 gm/ml solution dissolved in 60 ml of water). One hour later, the senior author repeated the examination and videotaping. Patients were maintained on a dose of 1 gm twice per day (taken four to five hours apart, typically after breakfast and lunch) until their next office visit two weeks later, when the examination and videotaping were repeated one hour after receiving 2 gm of sodium oxybate by mouth (4 ml of 0.5 gm/ml solution dissolved in 60 ml of water). After two weeks taking 2 gm twice daily, the procedure was repeated after a 3 gm office dose, and finally two weeks after receiving 3 gm twice daily, the procedure was repeated after a 4 gm office dose. The maximum dose allowed in the trial was 4 gm twice daily. Patients and the senior author determined at each visit whether or not to proceed to the next dose level, based principally on their ability to tolerate the most recent dose regimen. After deciding on a maximum tolerated dose, patients received a dose in the office 0.5 gm less and the examination was videotaped.

Methods/Data Analysis

The entire videotape segments and patient-writing samples from each visit were copied, and were blinded to trial order and identifying features, and were randomly ordered for review using a random number table. A movement disorder expert blinded to trial design and dose schedule, scored each videotape. Sub-scores for each visit were calculated as described previously. Frucht S. J., et al. Adv Neurol 89:361-76 (2002). We modified section 5 of the UMRS in which functional performance (pouring water, using a soup spoon) is performed only with the dominant arm (section V). In this modification these tasks were videotaped while being performed with both arms, because myoclonic jerks were significantly worse in the non-dominant left arm in patients #2 and 3. This increased the maximum score of section 5 of the UMRS from 20 to 28.

Results

Tolerability: Transient headache and dizziness were common and did not require dose reduction (Table 2). All patients experienced dose-limiting sedation or emotional liability, however the dose at which this occurred varied from 2 to 4 gms between patients. These side effects resolved for each patient when the individual dose was reduced by 0.5 gm.

TABLE 2
Tolerability and dose regimens of Xyrem ®

| | | | | Dose @ | Maximum | |
|---|---|---|---|---|---|---|
| PT # | MILD SE | SER SE | D-L-SE | D-L-SE | trial dose | Final regimen |
| 1 | HA | asthma | sedation and mild | 3 gm | 2.5 gm BID | 2 gm TID |
| | (3 gm) | | disinhibition | | | |
| 2 | Ø | Ø | mild sedation and | 3 gm | 2.5 gm BID | 2.5 gm q AM |
| | | | emotional lability | | | 2.5 gm q PM |
| | | | | | | 2.5 gm qhs |
| 3 | HA (1 gm) | Ø | slight sedation | 4 gm | 3.5 gm BID | 3.5 gm BID |
| | dizzy (1 gm) | | | | | |
| 4 | HA (1 gm) | Ø | slight sedation and | 2 gm | 1.5 gm BID | 1.5 gm BID |
| | dizzy (2 gm) | | emotional lability | | | |
| 5 | Ø | Ø | slight sedation | 2 gm | 1.5 gm BID | 1.5 gm BID |

SE—side effect;
Ser—serious SE (causing illness leading to hospitalization);
D-L-SE: dose-limiting side effect

One serious adverse event occurred during the trial. Patient #1 developed an upper respiratory infection that triggered an asthma exacerbation, requiring treatment with oral antibiotics, prednisone and frequent bronchodilator inhalers. As similar events had occurred in the past, the senior author judged that this event was not likely related to the study drug and she was continued in the trial. Myoclonus visibly worsened during her asthma exacerbation (her third office visit at which time she received 3 gm of sodium oxybate), but by the next visit the respiratory infection had resolved, steroids and antibiotics had been discontinued, and myoclonus had improved.

Clinical course: Improvement in involuntary movements was dose-dependent, and could be observed in the office by the patient and senior author within 30 to 45 minutes after receiving each dose. The duration of benefit was 3½ to 4 hours, and as patients titrated to higher doses they became aware when the dose would wear off. Patients described the benefit of treatment as similar to the effect of ethanol. Dose-limiting sedation roughly correlated with the maximum amount of ethanol that patients could tolerate. We did not observe a waning of effectiveness of the drug during the course of the trial. All five patients decided to continue taking the drug after completing the trial, and due to the four-hour duration of action, dosing schedules were adjusted for patients #1 and 2.

Blinded Rating of Efficacy:

Myoclonus Patients (#s 1-3): In three myoclonus patients, myoclonus at rest (section 2 of the UMRS) and stimulus-sensitive myoclonus (section 3) improved in dose-dependent fashion, (Table 3 a-c). Action myoclonus (section 4) improved by 50%, 57% and 88% respectively, while functional performance (section 5) improved by 40%, 60% and 25% (videotape segments 1-3). Patient self-assessment scores improved for patients #2 and 3, and were unchanged for patient #1. Physician global assessment scores (UMRS part 6) were “mild” (1 out of 4) for patients #2 and 3 and remained unchanged throughout the trial, while scores decreased from severe disability (4) to moderate impairment (2) in patient #1.

Severe action myoclonus was observed to prevent patient #1 from putting pen to paper or targeting a spoon to a cup. One hour after receiving 2.5 gm of sodium oxybate, she was able to write (although slowly) and her control of the spoon was improved. Writing, pouring and using a spoon in patient #2, triggered proximal and axial flurries of myoclonus. After receiving 3 gm of sodium oxybate, the amplitude and frequency of the jerks was diminished and the movements were more fluid. In patient #3, walking triggered myoclonic jerks of the right leg, and violent proximal and truncal myoclonus was activated with writing and pouring. After receiving 4 gm of sodium oxybate, his walking was modestly improved. Although the blinded review of his functional performance scores (section 5 of the UMRS) were unchanged, writing and pouring appeared modestly improved.

Essential tremor scores (patients #4, 5): Blinded videotape review revealed dose-dependent improvement in sustention tremor and action tremor (Table 3d and e) of 79% in patient #4, and 48% in patient #5. Scores for rest tremor were not calculated, as rest tremor was absent in one patient and mild in the other. Blinded rating of the severity of torticollis in patient #4 decreased from “moderate” to “mild” at the 1 gm twice-daily dose. Patient #4's examination before treatment revealed a classic kinetic tremor with writing, using a spoon, and with drinking. After 2 gm of sodium oxybate, tremor amplitude was markedly diminished. Patient #5's kinetic tremor on pouring, using a spoon, and drinking were more severe. Although still present after receiving 2 gm of sodium oxybate, the amplitude has diminished and voluntary movements are more fluid.

TABLE 3 A-E: Blind Ratings of UMRS and WHIGET Scores for Patients

a: UMRS sub scores for patient 1

| UMRS | Score | | | | | | Rx |
|---|---|---|---|---|---|---|---|
| section # | range | 0 gm | 1 gm | 2 gm | 3 gm | 2.5 gm | effect |
| 1 | 0-44 | 33 | NM | NM | 31 | NM | |
| 2 | 0-128 | 3 | 3 | 0 | 3 | 0 | |
| 3 | 0-17 | 8 | 3 | 1 | 3 | 2 | |
| 4 | 0-160 | 108 | 96 | 64 | 86 | 54 | 50% |
| 5 | 0-20 | 20 | 20 | 13 | 15 | 12 | 40% |
| 6 | 0-4 | 4 | 3 | 3 | 3 | 2 | |

b: UMRS sub scores for patient 2

| UMRS | Score | | | | | | Rx |
|---|---|---|---|---|---|---|---|
| section # | range | 0 gm | 1 gm | 2 gm | 3 gm | 2.5 gm | effect |
| 1 | 0-44 | 5 | NM | NM | NM | 0 | |
| 2 | 0-128 | 4 | 2 | 3 | 0 | 0 | |
| 3 | 0-17 | 4 | 2 | 2 | 0 | 0 | |
| 4 | 0-160 | 25 | 29 | 9 | 11 | 3 | 88% |
| 5 | 0-28 | 10 | 8 | 4 | 5 | 4 | 60% |
| 6 | 0-4 | 1 | 1 | 1 | 1 | 1 | |

c: UMRS sub scores for patient 3

| UMRS | Score | | | | | Rx |
|---|---|---|---|---|---|---|
| section # | range | 0 gm | 1 gm | 2 gm | 4 gm | effect |
| 1 | 0-44 | 10 | NM | 7 | 5 | |
| 2 | 0-128 | 15 | 13 | 15 | 10 | |
| 3 | 0-17 | 6 | 2 | 3 | 1 | |
| 4 | 0-160 | 35 | 25 | 26 | 15 | 57% |
| 5 | 0-28 | 12 | 13 | 9 | 12 | 25% |
| 6 | 0-4 | 1 | 1 | 1 | 1 | |

d: WHIGET sub scores for patient 4

| | | | | | | Rx |
|---|---|---|---|---|---|---|
| WHIGET section | Score range | 0 gm | 1 gm | 2 gm | 1.5 gm | effect |
| TOTAL | 0-6 | 2.5 | 0.5 | 0 | 0.5 | |
| SUSTENTION | | | | | | |
| TOTAL ACTION | 0-40 | 19.5 | 10 | 5 | 4 | 79% |

e: WHIGET sub scores for patient 5

| | | | | | | Rx |
|---|---|---|---|---|---|---|
| WHIGET section | Score range | 0 gm | 1 gm | 2 gm | 1.5 gm | effect |
| TOTAL | 0-6 | 4 | 3 | 3 | 2.5 | |
| SUSTENTION | | | | | | |
| TOTAL ACTION | 0-11 | 23 | 19 | 12 | 13.5 | 48% |

Discussion

In this open-label trial, sodium oxybate produced dose-dependent improvements in blinded ratings of ethanol-responsive myoclonus and tremor. The drug was tolerated at doses that produced clinical benefit. The most common side effect was sedation, which was also dose-dependent, however the dose that produced clinical benefit was lower than the sedation-limiting dose.

Xyrem® is currently approved in the United States only for treatment of cataplexy in narcoleptic patients. All patients who receive Xyrem® must be enrolled in the Xyrem® Success Program, a central registry that monitors and distributes the drug. Fuller, D. E., et al. Drug Saf 27:293-306 (2004). The Xyrem® Success Program has ensured appropriate and safe use of the drug with no incidents of diversion or inappropriate use. Stahl P., et al. Sleep 27(suppl): A247 (2004). Sodium oxybate should not be used in patients with movement disorders outside of a protocol approved by a medical center's institutional review board. These protocols should include videotaped examinations or placebo-controlled designs using validated clinical rating scales. Patient selection is critical, and patients with a history of active substance abuse, poor compliance or major depression should be excluded from participation. This is of particular concern in MD patients where there is an increased risk of ethanol abuse, and also in patients with intractable hyperkinetic movement disorders who might adjust their dosing regimens in a search for therapeutic benefit.

The mechanism of sodium oxybate's anti-myoclonic and anti-tremor activity remains unknown. Gamma-hydroxybutyric acid (GHB) occurs naturally in the brain and is formed through metabolism of its precursor, gamma-aminobutyric acid (GABA). Waszkielewicz, A., et al. Pol J Pharmacol 56:43-9 (2004). The GHB receptor is distinct from the GABA-B receptor and when given as a drug, it is likely that some GHB is converted to GABA. Wu, Y. et al. Neuropharmacology 47:1146-56 (2004); (Waszkielewicz, A., et al. Pol J Pharmacol 56:43-9 (2004). Sodium oxybate may act via the GABA-B receptor, either directly or via conversion to GABA. Kaupmann, K., et al. Euro J Neuro 18:2722-2730 (2003). However GABA-B agonists such as baclofen do not improve ET or myoclonus, and clonazepam has minimal effect on ET, suggesting that other mechanisms may be involved.

Because our trial was open-label, placebo effect limits broader application of the data to other patients, and also likely contributed to the perception of benefit by patients' #1-3 (section 1 of the UMRS). However, some lessons may be learned from our experience. Patient #1 is similar to our prior patient with ethanol-responsive PHM. Frucht, S. J., et al. Mov Disord 20:1330-7 (2005). Prominent stimulus-sensitive proximal jerks and postural negative myoclonus suggests a pattern consistent with reticular reflex myoclonus in both cases. Hallett, M., et al. J Neurol Neurosurg Psychiatry 40:253-64 (1977). Reticular reflex PHM is sufficiently rare that a double blind, placebo-controlled trial of sodium oxybate in this patient population may not be feasible. It therefore seems reasonable to consider a test dose of ethanol in these patients if standard anti-myoclonic drugs fail. Patients who respond to ethanol might also benefit from treatment with sodium oxybate. Myoclonus also improved in our two patients with MD, a finding similar to Priori's observation. Priori, A., et al. Neurology 54:1706 (2000). However given the risk of ethanol abuse in the MD population, the long-term tolerability of sodium oxybate must be established before it can be recommended as a treatment for MD patients.

Present treatments for ET include primidone, propranolol, gabapentin, levetiracetam, topiramate, and 1-octanol. Findley, L. K., et al. J Neurol Neurosurg Psychiatry 48:911-5 (1985); Baruzzi, A., et al. Neurology 33:296-300 (1983); Ondo, W., et al. Mov Disord 15:678-382 (2000); Handforth, A., et al. Mov Disord 19:1215-21(2004); Connor, G. S. et al. Neurology 59:132-4 (2002); Shill, H. A., et al. Neurology 62:2320-2(2004). Deep brain stimulation (DBS) of the ventrointermediate thalamus is currently the most reliable technique for producing immediate relief of appendicular tremor. Vaillancourt, D. E., et al. Neurology 61:919-25 (2003). Bilateral stimulation is typically required for head tremor and voice tremor, and the unavoidable but small operative risks of DBS and the possibility of delayed lead failure or infection are a concern. Berk, C., et al. J Neurosurg 96:615-8 (2002); Yoon, M. S., et al. Stereotact Funct Neurosurg 72:241-4 (1999); Binder, D. K., et al. Steroetact Funct Neurosurg 80:28-31 (2003); Kondziolka, D., et al. Stereotact Funct Neurosurg 79:228-33 (2002).

This invention provides for methods of treating patients with other alcohol-responsive movement disorders with the compound of Formula I and other compounds of the invention. The invention provides also for methods to assess whether movement disorders that do not benefit from ethanol (for example half of all patients with ET) will benefit from treatment. If not, then the response to the drug may reveal potential differences in pathogenesis between responsive and non-responsive patients.

In an open-label pilot tolerability and efficacy study of five patients with ethanol-responsive movement disorders, we have shown that sodium oxybate improved myoclonus and tremor and those patients were able to tolerate daytime dosing of the drug. Further studies of this agent in patients with hyperkinetic movement disorders are warranted.

### Example 4

### Clinical Trial

Twenty patients age 18 or older were recruited from a clinical practice at the Movement Disorders Division of Columbia University Medical Center. Eligible patients with medication-refractory, ethanol-responsive myoclonus (6 PHM, 3 MD, 2 PME) or ET (9 patients) (Table 1) were offered enrollment from February 2004 to March 2005. The medical center's institutional review board approved the protocol, and written informed consent was obtained from all participants. All had myoclonus or ET that improved (by self-report) with ingestion of ethanol. All patients were medication-refractory, defined as obtaining inadequate benefit from best medical treatment or being unable to tolerate treatment. The dose and timing of other medications were kept constant before and during the trial.

TABLE 1
Table 1: Clinical features of the twenty patients are summarized in this table.

| PT.# | DX | AGE | SX | Current meds | Past meds | RX? | DOSE (GM) |
|---|---|---|---|---|---|---|---|
| 1 | MD | 25 | 12 | levetiracetam | clonazepam, primidone | Y | 7 |
| | | | | | trihexyphenidyl | | |
| 2 | MD | 20 | 17.5 | clonazepam | baclofen, L-5-HTP | Y | 7 |
| | | | | | levetiracetam, trihexyphenidyl | | |
| | | | | | valproic acid | | |
| 3 | MD | 21 | 10 | Ø | clonazepam, levetiracetam | Y | 9 |
| 4 | PME | 64 | 19 | clonazepam, levetiracetam, | tiagabine, L-5-HTP | Y | 7.5 |
| | | | | valproic acid | phenobarbital, piracetam | | |
| 5 | PME | 35 | 24 | acetazolamide, levetiracetam | felbamate, L-5-HTP | Y | 6 |
| | | | | primidone | phenobarbital, valproic acid | | |
| 6 | PHM | 38 | 4.5 | clonazepam | phenobarbital, topiramate | Y | 9 |
| | | | | levetiracetam | valproic acid, zonisamide | | |
| 7 | PHM | 37 | 6 | clonazepam, levetiracetam, | piracetam, primidone | Y | 7.5 |
| | | | | phenobarbital | valproic acid | | |
| 8 | PHM | 62 | 0.75 | clonazepam, levetiracetam | Ø | N | 3 |
| | | | | zonisamide | | | |
| 9 | PHM | 62 | 23 | clonazepam, valproic acid | levetiracetam | N | 4.5 |
| 10 | PHM | 65 | 2.5 | clonazepam, levetiracetam | valproic acid | N | 3 |
| | | | | zonisamide | | | |
| 11 | PHM | 54 | 16 | clonazepam, levetiracetam | L-5-HTP, phenobarbital, | Y | 7.5 |
| | | | | | piracetam, topiramate, | | |
| | | | | | valproic acid | | |
| 12 | ET | 67 | 50 | Ø | Ø | N | 3 |
| 13 | ET | 75 | 14 | levetiracetam | primidone | N | 3 |
| 14 | ET | 76 | 20 | primidone | propranolol | Y | 1.5 |
| 15 | ET | 73 | 20 | Ø | primidone, propranolol | Y | 4.5 |
| 16 | ET | 62 | 2 | Ø | Ø | Y | 4.5 |
| 17 | ET | 63 | 45 | Ø | primidone, propranolol | Y | 6 |
| 18 | ET | 81 | 65 | primidone, propranolol | Ø | Y | 7.5 |
| 19 | ET | 71 | 12 | propranolol, topiramate | primidone | N | 3 |
| 20 | ET | 73 | 12 | Ø | primidone, propranolol | Y | 6 |

Dx: diagnosis;
Age: in years at time of enrollment:
Sx: symptom duration in years at time of enrollment;
Current meds: continued at time of enrollment;
Past meds: medications taken in the past for treatment of involuntary movements (excluding those currently being administered);
Rx?: decision (Yes or No) to continue treatment with sodium oxybate after completion of the trial;
Dose: total daily dose of sodium oxybate on completion of the trial.

Twelve men and 8 women enrolled. Results from patients #6, and #s 1, 2, 7, 12, and 13 were reported previously. Frucht, S. J., et al. Mov Disord 20:745-51 (2005); Frucht, S. J., et al. Mov Disord 20:1330-7 (2005). Mean age/symptom duration was 43.9 yrs/12.3 yrs (myoclonus) and 71.2 yrs/26.7 yrs (ET). Patients with myoclonus were examined and videotaped at each visit using the Unified Myoclonus Rating Scale (UMRS), and patients with ET were examined and videotaped using the modified Washington Heights Inwood Genetic Essential Tremor Rating Scale (WHIGET; Appendix 1). Frucht, S. J., et al. Mov Disord 20:1330-7 (2005). After initial baseline examination and videotaping, patients took 1 gm of sodium oxybate by mouth (2 ml of the standard 0.5 gm/ml solution dissolved in 60 ml of water), and one hour later the videotaped examination was repeated. Patients were maintained on a dose of 1 gm T.I.D. (taken before meals) until their next office visit two weeks later, when the examination and videotaping were repeated one hour after receiving 1.5 gm of sodium oxybate. Subsequent visits and dose titrations at two-week intervals were repeated until the maximum dose was reached (3 gm T.I.D), patients were satisfied with the results of treatment, or until they developed side effects that they viewed as troublesome. Dosing for patients #1, 2, 6, 7, 12, and 13 was slightly different (1 gm BID, with dose increments of 2 gm to maximum dose of 4 gm BID), but office visits and videotaping protocol were otherwise identical.

APPENDIX 1
Unified Myoclonus Rating Scale (UMRS) SCORE SHEET
1. PATIENT QUESTIONNAIRE

| | |
|---|---|
| A. (Speech) | {0-4} |
| B. (Reading) | {0-4} |
| C. (Handwriting) | {0-4} |
| D. (Eating) | {0-4} |
| E. (Drinking) | {0-4} |
| F. (Swallowing) | {0-4} |
| G. (Hygiene) | {0-4} |
| H. (Dressing) | {0-4} |
| I. (Arising) | {0-4} |
| J. (Standing) | {0-4} |
| K. (Walking) | {0-4} |
| TOTAL | {0-44} |
| PT. GLOBAL ASSESSMENT | {0-4} |

2. MYOCLONUS AT REST

| Body Part | Frequency | | Amplitude | |
|---|---|---|---|---|
| Upper Face | | × | | {0-16} |
| Lower Face | | × | | {0-16} |
| Neck | | × | | {0-16} |
| Trunk | | × | | {0-16} |
| R Arm | | × | | {0-16} |
| L Arm | | × | | {0-16} |
| R Leg | | × | | {0-16} |
| L Leg | | × | | {0-16} |
| | | | TOTAL 1108 | {0-108} |

3. STIMULUS SENSTIVITY.
Score 1 if a stimulus produces a jerk in any body part;
score 0 if no jerk is elicited. Each stimulus is performed only once.

| Threat: thrust hands towards patient's face unexpectedly: | | |
|---|---|---|
| Claps hands unexpectedly: | | |
| Tap patient's nose with patient's eyes closed: | | |
| Elicit jaw jerk: | | |
| Pin prick: | On cheek | |
| | R arm (flexor surface of wrist) | |
| | L arm (flexor surface of wrist) | |
| | R leg (bottom of foot) | |
| | L leg (bottom of foot) | |
| Finger flick: | R index finger | |
| | L index finger | |
| Toe flick: | R great toe | |
| | L great toe | |
| Reflexes: | R bicep | |
| | L bicep | |
| | R knee jerk | |
| | L knee jerk | |
| | STIMULUS-SENSITIVITY TOTAL | {0-17} |

4. MYOCLONUS WITH ACTION

| Action | Frequency | | Amplitude | |
|---|---|---|---|---|
| A. Close Eyelids | | × | | {0-16} |
| B. Neck | | × | | {0-16} |
| C. Trunk | | × | | {0-16} |
| D. R arm | | × | | {0-16} |
| E. L arm | | × | | {0-16} |
| F. R leg | | × | | {0-16} |
| G. L leg | | × | | {0-16} |
| H. Arising | | × | | {0-16} |
| I. Standing | | × | | {0-16} |
| J. Walking | | × | | {0-16} |
| | | | TOTAL | {0-160} |

5. FUNCTIONAL TESTS

| | |
|---|---|
| A. Writing | {0-4} |
| B. R hand spiral | {0-4} |
| C. L hand spiral | {0-4} |
| D. R pouring water | {0-4} |
| E. L pouring water | {0-4} |
| F. R soup spoon | {0-4} |
| G. L soup spoon | {0-4} |
| TOTAL | {0-28} |

| | |
|---|---|
| 6. GLOBAL DISABILITY SCORE | {0-4} |

Sections 1-6 of the UMRS appear above. Full details regarding the scale are available in reference 16. Higher scores indicate more severe involuntary movements. The range for each section appears in brackets { }.

Ratings and Data Analyses

The entire videotape segments and writing samples from each visit were copied and randomly ordered, and identifying features that might reveal trial order or drug dose were removed. A blinded movement disorder neurologist scored each videotape segment using the UMRS or the WHIGET. Sub-scores for each visit were calculated as described previously. Frucht, S. J., et al. Mov Disord 20:1330-7 (2005). Based on data from the first two ET cases, and assuming alpha=0.05, power=80%, and a 25% reduction in post-treatment tremor severity, we calculated that eight ET cases were required. A similar number of myoclonus patients would provide >80% power to detect a 25% reduction in the severity of post-treatment action myoclonus. Paired t tests were performed for scores pre- and post-treatment.

Results

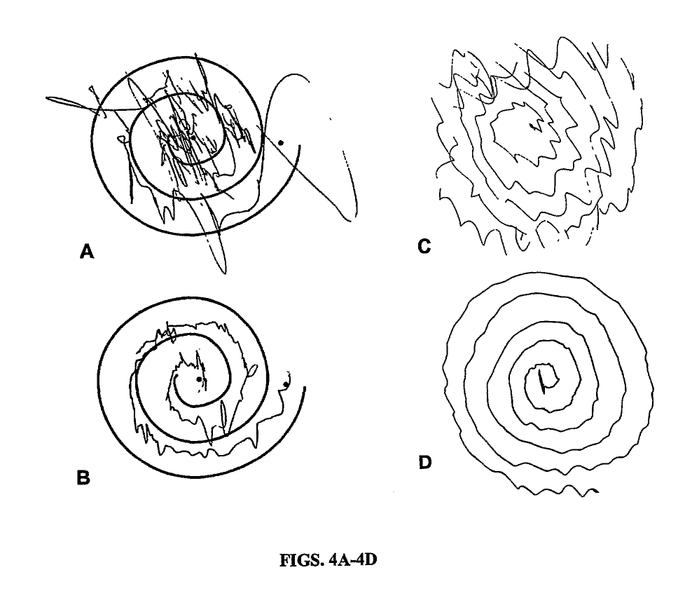

A patient with ET (patient #15) was observed and video-recorded pouring water from one cup to another at 15-minute intervals after receiving 1.5 gm of sodium oxybate in the office. Improvement in action tremor during pouring was evident at 45 minutes and obvious at 60 minutes after treatment. Prior to treatment, tremor during tasks (drawing and sipping water from spoon) was also evident in patient #17 displaying ET (FIG. 4). Improvement in kinetic tremor during tasks was evident after being treated with sodium oxybate (FIG. 4).

Myoclonus at rest was present in patients #3 (MD) and #11 (PHM) before treatment and disappeared after receiving 3 mg and 2.5 mg of sodium oxybate, respectively. In patient #4 displaying PME, stimulus-sensitive myoclonus to sound and pinprick was present prior to treatment. After receiving 3 gm of sodium oxybate, only stimulus-sensitive myoclonus of the right hand remained. Action myoclonus on finger-to-nose and negative myoclonic postural lapses was severe in patient #6 (PHM) before receiving sodium oxybate. While still present after treatment, action myoclonus was significantly improved, and standing unassisted was now possible in patient #6. One measure of functional performance, using a soupspoon, improved with receiving sodium oxybate in patients #9 (PHM) and #5 (PME).

The severity of myoclonus at rest, stimulus-sensitive myoclonus, action myoclonus and functional performance decreased, as did mean postural and kinetic tremor scores (Table 2, FIG. 4). Maximal improvement in these measures occurred at doses just below the highest dose employed in the trial (2.5 gm for myoclonus, 1.5 gm for ET).

TABLE 2
Table 2: Kinetic and postural tremor scores at various individual doses of sodium oxybate are
displayed in the second and third columns from the left.

| Individ. | Myoclonus | Stim-sens. | Action | Functional | Individ. | Kinetic | Postural |
|---|---|---|---|---|---|---|---|
| dose | at rest | myoclonus | myoclonus | performance | dose | tremor | tremor |
| (gm) | score (p) | score (p) | score (p) | score (p) | (gm) | score (p) | score (p) |
| 0 | 7.4 | 5.8 | 45.3 | 12.9 | 0 | 15.4 | 2.4 |
| 1 | 4.9 (0.137) | 3.2 (0.01)** | 33.4 (0.047)* | 11.0 (0.044)* | 1 | 11.5 (0.011)* | 1.7 (0.029)* |
| 1.5 | 1.2 (0.247) | 3.2 (0.11) | 26.8 (0.122) | 9.6 (0.007)* | 1.5 | 8.8 (0.006)** | 1.1 (0.001)** |
| 2 | 3.6 (0.047)* | 2.8 (0.001)** | 30.9 (0.005)* | 6.0 (<0.001)** | 2 | 9.1 (0.006)** | 1.4 (0.006)** |
| 2.5 | 0.6 (0.055) | 1.4 (<0.001)** | 26.2 (0.046)* | 7.4 (0.002)** | | | |
| 3.0 | 0.75 (0.129) | 1.0 (0.02)* | 33.0 (0.064) | 6.9 (0.059) | | | |

Scores at various individual doses of sodium oxybate for myoclonus at rest, stimulus-sensitive myoclonus, action myoclonus and functional performance are displayed in the fifth through the eight columns from the left.
P values are displayed in italics for each score relative to pre-treatment values:
values ≦0.05 are noted with the symbol *, and
values ≦0.01 with the symbol **.

The average final daily dose of sodium oxybate for patients with myoclonus was 6.5 gm, (range 3-9 gm) and 4.3 gm for ET, (range 1.5-7.5 gm). Mild, transient side effects included dizziness (35%), headache (20%), emotionality (20%), and nausea (10%). Dose titration was stopped in two patients due to adequate benefit, or was limited by sedation (60%) or ataxia (20%). These side effects resolved when the dose was reduced to the previous level. Fourteen patients chose to continue the drug after completing the trial.

Discussion

Blinded ratings of myoclonus and tremor decreased with sodium oxybate therapy in this trial of twenty patients with medication-refractory hyperkinetic movements. Tolerability of daytime dosing was acceptable, and the majority of patients chose to continue treatment after completing the trial.

We are aware of the limitations of open-label design, including the likelihood that patients refractory to conventional treatment are predisposed to experience placebo benefit. Proof of efficacy of sodium oxybate as a treatment for these disorders will require double blind, placebo-controlled trials. Patient selection for these trials will be important, as individuals with a history of substance abuse, depression, non-compliance, or a tendency to adjust their medication dosing should not take this drug. We did not include quality of life or functional performance measures in the current study beyond those contained in the WHIGET or UMRS scales; these measures will be important for future trial design.

We believe that several factors support a biologic treatment effect for the drug. One compelling argument is that the majority of patients decided to continue the drug after the trial. Another is that as patients titrated to higher doses, most became aware of the drug's onset of action 45 to 60 minutes after ingestion, and its tendency for benefit to wear-off in four to five hours. Increasing benefit in blinded myoclonus and tremor scores was observed as the dose increased. The most significant improvements were seen at doses just below the maximum. Mild worsening of scores occurred at the highest doses employed in several patients. One possible explanation for this effect is that the highest doses unmasked cerebellar deficits in these patients, slightly impairing their performance.

The mechanism of action of sodium oxybate in myoclonus and tremor remains unknown, although a GABA-ergic mechanism is possible. Mice deficient in the GABA_{A} receptor exhibit an essential-like tremor that is completely inhibited by ethanol, implying that GABA-ergic mechanisms may be important in ET. Kralic, J. E. et al. J Clin Invest 115:774-779 (2005). Alternatively, sodium oxybate may restore motor networks in these patients to a normal state, for example normalizing ventrolateral thalamic activation in PHM or bilateral cerebellar hemispheric activation in ET. Frucht, S. J., et al. Neurology 62:1879-1881(2004); Boecker, H. et al. Ann Neurol 39:650-658 (1996).

While the foregoing invention has been described in some detail for purposes of clarity and understanding, these particular embodiments are to be considered as illustrative and not restrictive. It will be appreciated by one skilled in the art from a reading of this disclosure that various changes in form and detail can be made without departing from the true scope of the invention.

What is claimed is:

1. A method for treating myoclonus comprising administrating to a patient a compound of formula (I): 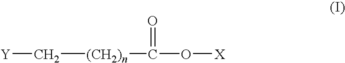
wherein n is 1-2, X is H, a pharmaceutically acceptable cation or (C₁-C₄)alkyl, and Y is OH, (C₁-C₄)alkoxy, CH(Z)CH₃, (C₁-C₄)alkanoyloxy, phenylacetoxy or benzoyloxy or where X and Y are connected as a single bond,
wherein Z is OH, (C₁-C₄)alkoxy, (C₁-C₄)alkanoyloxy, phenylacetoxy or benzoyloxy,
wherein the myoclonus is not alcohol-sensitive essential myoclonus with dystonia.

2. The method of claim 1, wherein the patient exhibits one or more of the following: negative myoclonus, myoclonus at rest, stimulus-sensitive myoclonus, or action myoclonus.

3. The method of claim 1, further comprising administering to the patient a second anti-myoclonic agent.

4. The method of claim 1, wherein the second anti-myoclonic agent is selected from clonazepam, levetiracetam, valproic acid, phenobarbital, topiramate, and zonisamide.

5. A method for ameliorating negative myoclonus comprising administrating to a patient a compound of formula (I).

6. A method for ameliorating myoclonus at rest comprising administrating to a patient a compound of formula (I).

7. A method for ameliorating stimulus-sensitive myoclonus comprising administrating to a patient a compound of formula (I).

8. A method for ameliorating action myoclonus comprising administrating to a patient a compound of formula (I).

9. The method of claim 5, 6, 7, or 8, wherein the amelioration is assessed by use of the Unified Myoclonus Rating Scale.

10. The method of claim 5, 6, 7, or 8, wherein the amelioration is assessed by use of the Chadwick-Marsden Scale.

11. A method for improving the functional performance of a patient diagnosed with myoclonus comprising administrating to a patient a compound of formula (I).

12. The method of claim 11, wherein the improvement is assessed by use of the Unified Myoclonus Rating Scale.

13. The method of claim 11, wherein the improvement is assessed by use of the Chadwick-Marsden Scale.

14. A method for treating myoclonus comprising administrating to a patient sodium oxybate, wherein the myoclonus is not alcohol-sensitive essential myoclonus with dystonia.

15. A method for treating myoclonus comprising administrating to a patient sodium gamma-hydroxybutyrate, wherein the myoclonus is not alcohol-sensitive essential myoclonus with dystonia.

16. A method for treating essential tremor comprising administrating to a patient a compound of formula (I): 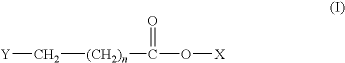
wherein n is 1-2, X is H, a pharmaceutically acceptable cation or (C₁-C₄)alkyl, and Y is OH, (C₁-C₄)alkoxy, CH(Z)CH₃, (C₁-C₄)alkanoyloxy, phenylacetoxy or benzoyloxy or where X and Y are connected as a single bond, wherein Z is OH, (C₁-C₄)alkoxy, (C₁-C₄)alkanoyloxy, phenylacetoxy or benzoyloxy.

17. The method of claim 1 or 16, wherein Y is OH or (C₁-C₄)alkanoyloxy.

18. The method of claim 17, wherein X is a pharmaceutically acceptable cation.

19. The method of claim 18, wherein X is Na⁺.

20. The method of claim 1 or 16, wherein Y is OH and X is Na⁺.

21. The method of claim 1 or 16, wherein X is H or a pharmaceutically acceptable cation and Y is OH.

22. The method of claim 1 or 16, wherein the compound of formula (I) is γ-butyrolactone.

23. The method of claim 16, wherein the patient exhibits one or more of the following: benign tremor, postural tremor, or kinetic tremor.

24. The method of claim 16, further comprising administering to the patient a second anti-tremor agent.

25. The method of claim 16, wherein the second anti-tremor agent is selected from the groups comprising mysoline, propranolol, gabapentin, levetiracetam, and topiramate.

26. The method of claim 1 or 16, wherein a daily dose of about 1 to 500 mg/kg is administered.

27. The method of claim 1 or 16, wherein a daily dose of about 500 mg to about 20 g is administered.

28. The method of claim 1 or 16 wherein a daily dose of about 2-10 g is administered.

29. The method of claim 1 or 16, wherein a dose of about 1-5 g is administered twice daily.

30. The method of claim 28 or 29, wherein sodium oxybate is administered.

31. The method of claim 1 or 16, wherein the compound of formula (I) is administered orally, in combination with a pharmaceutically acceptable carrier.

32. The method of claim 31, wherein the compound of formula (I) is sodium gamma-hydroxybutyrate

33. The method of claim 31, wherein the carrier is a liquid.

34. The method of claim 31, wherein the carrier is a tablet or capsule.

35. The method of claim 1 or 16, wherein the compound of formula (I) is administered parenterally, in combination with a pharmaceutically acceptable carrier.

36. The method of claim 35, wherein the compound is administered by injection or infusion.

37. The method of claim 1 or 16, wherein the compound is administered by inhalation.

38. The method of claim 1 or 16, wherein the compound is administered by means of a transdermal patch.

39. The method of claim 1 or 16, wherein the compound of formula (I) is administered orally, in a prolonged release dosage form.

40. The method of claim 39, wherein the compound of formula (I) is administered in conjunction with a compound that inhibits its metabolism in vivo.

41. The method of claim 40, wherein the compound of formula (I) is administered by infusion.

42. The method of claim 1 or 16, wherein the patient is a mammal.

43. The method of claim 1 or 16, wherein the mammal is human, primate, mouse, or rat.

44. A method for ameliorating hand tremor comprising administrating to a patient a compound of formula (I).

45. A method for ameliorating arm tremor comprising administrating to a patient a compound of formula (I).

46. The method of claim 44 or 45, wherein the amelioration is assessed by use of the Collaborative Clinical Classification of Tremor.

47. The method of claim 44 or 45, wherein the amelioration is assessed by use of the Classification of Essential Tremor.

48. The method of claim 44 or 45, wherein the amelioration is assessed by use of the WHIGET scale.

49. A method for treating essential tremor comprising administrating to a patient sodium oxybate.

50. A method for treating essential tremor comprising administrating to a patient sodium oxybate.

51. Therapeutic method of treating a hyperkinetic movement disorder comprising administering to a human afflicted with a myoclonus an effective amount of a compound of formula (I): 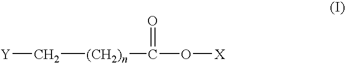
wherein n is 1-2, X is H, a pharmaceutically acceptable cation or (C₁-C₄)alkyl, and Y is OH, (C₁-C₄)alkoxy, CH(Z)CH₃, (C₁-C₄)alkanoyloxy, phenylacetoxy or benzoyloxy or where X and Y are connected as a single bond, wherein Z is OH, (C₁-C₄)alkoxy, (C₁-C₄)alkanoyloxy, phenylacetoxy or benzoyloxy, wherein the amount is effective to alleviate at least one symptom of said myoclonus, wherein said myoclonus is not alcohol-sensitive essential myoclonus with dystonia.

52. The method of claim 51 wherein the myoclonus is alcohol-responsive posthypoxic myoclonus.

53. The method of claim 51 wherein the myoclonus is palatal myoclonus.

54. The method of claim 51 wherein the myoclonus is a startle syndrome.

55. The method of claim 51 wherein the myoclonus is spinal myoclonus.

56. A therapeutic method of treating a hyperkinetic movement disorder comprising administering to a human afflicted with a dystonia, a tremor, or other hyperkinetic movement disorder, an effective amount of a compound of formula (I): 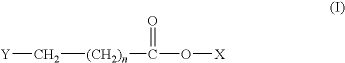
wherein n is 1-2, X is H, a pharmaceutically acceptable cation or (C₁-C₄)alkyl, and Y is OH, (C₁-C₄)alkoxy, CH(Z)CH₃, (C₁-C₄)alkanoyloxy, phenylacetoxy or benzoyloxy or where X and Y are connected as a single bond, wherein Z is OH, (C₁-C₄)alkoxy, (C₁-C₄)alkanoyloxy, phenylacetoxy or benzoyloxy, wherein the amount is effective to alleviate at least one symptom of said movement disorder.

57. The method of claim 56, wherein the movement disorder is a dystonia.

58. The method of claim 57, wherein the dystonia is a generalized dystonia.

59. The method of claim 57, wherein the dystonia is a focal dystonia.

60. The method of claim 56, wherein the tremor is essential tremor.

61. The method of claim 56, wherein the tremor is cerebellar tremor.

62. The method of claim 56, wherein the movement disorder is a tic.

63. The method of claim 56, wherein the movement disorder is ballismus.

64. The method of claim 56, wherein the movement disorder is chorea.

65. The method of claim 64, wherein chorea can be Huntington's disease.

66. The method of claim 51 or 56, wherein Y is OH or (C₁-C₄)alkanoyloxy.

67. The method of claim 66 wherein X is a pharmaceutically acceptable cation.

68. The method of claim 67 wherein X is Na⁺.

69. The method of claim 51 or 56, wherein Y is OH and X is Na⁺.

70. The method of claim 51 or 56, wherein the compound of formula (I) is γ-butyrolactone.

71. The method of claim 51 or 56, wherein the compound of formula (I) is administered orally, in combination with a pharmaceutically acceptable carrier.

72. The method of claim 71, wherein the compound of formula (I) is sodium gamma-hydroxybutyrate.

73. The method of claim 71, wherein the carrier is liquid.

74. The method of claim 71, wherein the carrier is a tablet or capsule.

75. The method of claim 69, wherein a daily dose of about 1-500 mg/kg is administered.

76. The method of claim 51 or 56, wherein a daily dosage of about 0.5-20 g is administered.

77. The method of claim 76, wherein sodium gamma-hydroxybutyrate is administered.

78. The method of claim 51 or 56, wherein the compound of formula (I) is administered orally, in a prolonged release dosage form.

79. The method of claim 78, wherein the compound of formula (I) is administered in conjunction with a compound that inhibits its metabolism in vivo.

80. The method of claim 51 or 56, wherein the compound of formula (I) is administered parenterally.

81. The method of claim 79, wherein the compound of formula (I) is administered by infusion.
